(19) United States
(12) Patent Application Publication
(10) Pub. No.: US 2009/0061417 A1
(43) Pub. Date: Mar. 5, 2009

(54) INFLUENZA A VIRUS DETECTION METHOD AND KIT THEREFORE

(75) Inventor: Masafumi Inoue, Singapore (SG)

Correspondence Address: DANN, DORFMAN, HERRELL & SKILLMAN, 1601 MARKET STREET, SUITE 2400, PHILADELPHIA, PA, 19103-2307, US

(73) Assignee: AGENCY FOR SCINECE TECHNOLOGY AND RESERCH, SINGAPORE (SG)

(21) Appl. No.: 12/093,780

(22) PCT Filed: Nov. 16, 2006
(86) PCT No.: PCT/SG06/00354

§ 371 Date: Oct. 2, 2008

(30) Foreign Application Priority Data
Nov. 16, 2005 (GB) 05233473.3

(51) Int. Cl. C12Q 1/70; C07H 21/00

(52) U.S. Cl. 435/5; 536/23.1

(57) ABSTRACT

The invention provides oligonucleotides for a simple, specific and/or sensitive test for the presence of Influenza A. In particular, the present invention provides a primer(s), probe(s) and/or test(s) for Influenza A Subtype H5N1. Kits comprising probe(s) and/or primer(s) useful in the test are also provided.

### FIELD OF THE INVENTION

The present invention relates to oligonucleotide(s), method(s) and kit(s) for influenza A virus infection detection. In particular, the invention provides a nucleic acid assay for the detection of Influenza A virus, subtype H5N1.

### BACKGROUND OF THE ART

Influenza A is an infectious disease of animals caused by type A strains of the influenza virus that normally infect birds, and less commonly, pigs. There are many subtypes of the influenza A virus. These subtypes are based on the haemagglutinin (HA) segment 4, which has 14 varieties and neuraminidase (NA) segment 6, which has 9 varieties. It is these two segments of the virus that cause virulence. Influenza A is an RNA virus of the Orthomyxovirus class.

Of the influenza A subtypes, H5N1 is of particular concern because it mutates quickly, has been shown to be highly pathogenic and can cause severe disease in man.

The spread and impact of Influenza A subtype H5N1 or avian flu has been well documented. From Southeast Asia, avian flu has spread to poultry in parts of China, Russia and now European countries like Turkey and Romania. Human cases of avian influenza have been confirmed in Hong Kong, Indonesia, Vietnam, Cambodia and Thailand.

Current laboratory methods of detecting influenza A virus infections commonly involve antigen detection, isolation in cell culture, or detection of influenza-specific RNA by reverse transcriptase-polymerase chain reaction (RT-PCR).

By using RT-PCR techniques with defined primers, it is possible to provide a viral detection as well as subtype identification. PCR primers for influenza A subtype H5N1 or avian flu have been designed by the University of Hong Kong during an outbreak in 1997 for the H5N1 strain of influenza A virus. However, Influenza A subtypes are prone to mutation so there is a high chance that any detection sequence chosen from the H5N1 sequence may change. This may decrease the detection sensitivity and specificity of any of the detection methods currently available.

The tests currently available are not sensitive and specific enough for simple detection methods; these risk having false positive results for influenza A subtype H5N1 or avian flu that could actually be due to other similar or related subtypes.

Accordingly, there is a need for a specific and sensitive detection method for influenza A virus infection. In particular, for a detection method specific for subtype H5N1 or avian flu.

### SUMMARY OF THE INVENTION

The present invention relates to a method and kit for determining the presence or absence of an Influenza type virus in a biological sample or from biological material isolated and/or purified from a biological sample.

The present invention provides a simple, sensitive and/or specific diagnostic test. By use of the probes and/or primers described herein, the method(s) and/or kit(s) are made more sensitive and/or specific than the detection methods of the prior art. Such tests may be by suspension array technology. Alternatively, such tests may be by a one step PCR method or a two step PCR.

The present invention provides an isolated oligonucleotide comprising at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragment(s) thereof, derivative(s) thereof, mutation(s) thereof, and complementary sequence(s) thereof. In particular, the present invention provides an isolated oligonucleotide comprising essentially of at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragment(s) thereof, derivative(s) thereof, mutation(s) thereof, and complementary sequence(s) thereof. More particularly, the present invention provides an isolated oligonucleotide consisting of a nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragment(s) thereof, derivative(s) thereof, mutation(s) thereof and complementary sequence(s) thereof, and complementary sequence(s) thereof. The isolated oligonucleotide may comprise at least one nucleotide sequence selected from the group consisting of SEQ ID NOS:30-54.

Accordingly, the present invention provides an isolated oligonucleotide comprising at least one nucleotide sequence comprising SEQ ID NO:21. The isolated oligonucleotide may comprise at least one nucleotide sequence selected from the group consisting of SEQ ID NO:30 and SEQ ID NO:31.

Accordingly, the present invention provides an isolated oligonucleotide comprising at least one nucleotide sequence comprising SEQ ID NO:22. The isolated oligonucleotide may comprise at least one nucleotide sequence selected from the group consisting of SEQ ID NO:32 and SEQ ID NO:33.

Accordingly, the present invention provides an isolated oligonucleotide comprising at least one nucleotide sequence comprising SEQ ID NO:16. The isolated oligonucleotide may comprise at least one nucleotide sequence of SEQ ID NO:34.

Accordingly, the present invention provides an isolated oligonucleotide comprising at least one nucleotide sequence comprising SEQ ID NO:26. The isolated oligonucleotide may comprise at least one nucleotide sequence selected from the group consisting of SEQ ID NO:35 and SEQ ID NO:36.

Accordingly, the present invention provides an isolated oligonucleotide comprising at least one nucleotide sequence comprising SEQ ID NO:18. The isolated oligonucleotide may comprise at least one nucleotide sequence selected from the group consisting of SEQ ID NOS:37 to 44.

Accordingly, the present invention provides an isolated oligonucleotide comprising at least one nucleotide sequence comprising SEQ ID NO:19. The isolated oligonucleotide may comprise at least one nucleotide sequence selected from the group consisting of SEQ ID NOS:45 to 48.

Accordingly, the present invention provides an isolated oligonucleotide comprising SEQ ID NO:20. The isolated oligonucleotide may comprise at least one nucleotide sequence selected from the group consisting of SEQ ID NOS:49 and 50.

Accordingly, the present invention provides an isolated oligonucleotide comprising at least one nucleotide sequence comprising SEQ ID NO:9. The isolated oligonucleotide may comprise at least one nucleotide sequence selected from the group consisting of SEQ ID NOS:51 and 52.

The present invention also provides a method of determining the presence of an H5N1 subtype of Influenza A virus in a biological sample, the method comprising the steps of:

(a) providing a biological sample;
(b) contacting at least one oligonucleotide with at least one nucleic acid in the biological sample, or contacting the oligonucleotide with at least one nucleic acid extracted, purified and/or amplified from the biological sample, wherein the oligonucleotide comprises at least one nucleotide sequence selected from the group consisting of:
SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragments(s) thereof, derivatives(s) thereof, mutation(s) thereof, and a complementary sequence(s) thereof;
and
(c) detecting any binding resulting from the contacting in step (b), whereby the detection of the binding indicates the presence of an H5N1 subtype of an Influenza A virus.

The oligonucleotide may comprise of at least one nucleotide sequence selected from the group consisting of SEQ ID NOS:30-54. The detecting in step (c) may comprise distinguishing between unbound oligonucleotide(s) and oligonucleotide(s) bound to the nucleic acid(s).

The oligonucleotide(s) may be immobilized on particles, for example, on microbeads. The oligonucleotides may be probes and the method comprising:

(i) providing a biological sample;
(ii) labeling the at least one nucleic acid in the biological sample or extracted, purified or amplified from the sample with at least one reporter label;
(iii) immobilizing at least one probe to at least one microbead comprising at least one fluorescent dye;
(iv) contacting the at least one probe with the at least one nucleic acid to allow binding of the probe(s) and nucleic acid(s);
(v) identifying microbeads based on the fluorescent intensity of the at least one fluorescent dye with a first laser light and detecting binding of nucleic acid(s) to probe(s) immobilized on identified microbead(s) with a second laser light based on the reporter label(s);
whereby the detection of binding of the nucleic acid(s) to probe(s) indicates the presence of the H5N1 subtype of Influenza A virus.

The at least one particle, (for example, a microbead) may comprise at least two fluorescent dyes. The at least two fluorescent dyes may be capable of allowing one microbead to be distinguished from another microbead based on the fluorescent intensities of the at least two fluorescent dyes. The labeling of the at least one nucleic acid in step (ii) may be done after the contacting in step (iv) and the step (c) of detecting may be carried out by using Suspension Array Technology.

According to one aspect of the present invention, the contacting in step (b) may comprise contacting at least two oligonucleotides forming a primer pair to the nucleic acid and the step (c) of detecting is by a polymerase chain reaction. In particular, the detecting may be by determination of the molecular weight of at least one amplicon obtained from the polymerase chain reaction. More in particular, the detecting may be by binding of at least one probe to at least one amplicon obtained from the polymerase chain reaction.

The primer pair is capable of binding to the nucleic acid(s) and amplify at least one amplicon comprising the sequence of SEQ ID NO:6. The primer pair may be a primer pair comprising at least one forward primer comprising the nucleotide sequence SEQ ID NO:25 and at least one reverse primer comprising the nucleotide sequence SEQ ID NO:26. In particular, the forward primer may comprise at least the nucleotide sequence of SEQ ID NO:34 and the reverse primer may comprise at least the one nucleotide sequence selected from the group consisting of SEQ ID NO:35 and SEQ ID NO:36.

The primer pair is capable of binding to the nucleic acid(s) and amplify at least one amplicon comprising the sequence of SEQ ID NO:7. The primer pair may be a primer pair comprising at least one forward primer comprising the nucleotide sequence SEQ ID NO:18 and at least one reverse primer comprising the nucleotide sequence SEQ ID NO:19. In particular, the forward primer may comprise at least one nucleotide sequence selected from the group consisting of SEQ ID NOS:37 to 44 and the reverse primer may comprise at least one nucleotide sequence selected from the group consisting of SEQ ID NOS:45 and SEQ ID NO:48. A probe comprising at least one sequence selected from the group consisting of SEQ ID NOS:49- and 50 may bind to the amplicon.

The primer pair is capable of binding to the nucleic acid(s) and amplify at least one portion of SEQ ID NO:7 the NA-300 gene. The primer pair may be a primer pair comprising at least one forward primer comprising the nucleotide sequence SEQ ID NO:8 and at least one reverse primer comprising the nucleotide sequence SEQ ID NO:9. In particular, the reverse primer may comprise at least one nucleotide sequence selected from the group consisting of SEQ ID NOS:51 and SEQ ID NO:52. A probe comprising SEQ ID NO:60 may bind to the amplicon.

The polymerase chain reaction may be followed by electrophoresis for detection and/or purification of the amplicon.

According to the method of the present invention, the contacting in step (b) and/or the binding in step (c) is for a time and under conditions sufficient for specific contacting or binding to occur between the oligonucleotide(s) (for example probes) and nucleic acid(s).

The biological sample may be from a human or non-human animal suspected to be infected with the Influenza B virus. The oligonucleotide may be labeled. The nucleic acid may be labeled.

The present invention also provides a kit for the detection of Influenza B virus, the kit comprising at least one oligonucleotide comprising a nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragment(s) thereof, derivative(s) thereof, mutation(s) thereof, and complementary sequence(s) thereof. In particular, the oligonucleotide may comprise at least one nucleotide sequence selected from the group consisting of SEQ ID NOS:30-54. The at least one oligonucleotide may be labeled. A kit for detection by suspension array technology may comprise at least one microbead, at least one fluorescent dye and/or at least one reporter label.

### BRIEF DESCRIPTION OF THE DRAWINGS

FIG. 1 shows a gel demonstrating the specificity of the primers of kit 2 of the present invention to the H5N1 subtype compared to other influenza A subtypes. Lane 1 is a marker, lane 2 H5N1 (−1) A/chicken/Vietnam/8/2004 (HPAI), lane 3 H5N3 (−1) A tern/Australia/75/(LPAI), lane 4 H7N3 (−1) A/chicken/queensland/1994 (HPAI) and lane 5 H7N7 (−1) A/duck/Victoria/1976 (LPAI). The primer that amplifies a region comprising SEQ ID 7 of NA may be approximately 300 bp while the primer that amplifies a region comprising SEQ ID NO: 10 of HA may be approximately 168 bp and the primer that amplifies a region comprising SEQ ID NO:1 of HA may be approximately 114 bp.

FIG. 2 shows the sensitivity achieved using primers according to the present invention for the detection of influenza A subtype H5N1 where the virus copy number per sample loaded varies from 500 copies per reaction to 0.5 copies per reaction. Lane 1 is a marker, lanes 2 & 3 contain 500 copies of the virus per 5 μl, lanes 4 & 5 contain 50 copies of the virus per 5 μl, lanes 6 & 7 contain 5 copies of the virus per 5 μl, lanes 8 & 9 contain 0.5 copies of the virus per 5 μl, lanes 10 contains a negative control of an unrelated virus. Positive DNA controls were prepared from individually cloned DNA derived from corresponding amplicons.

FIG. 3 shows a gel demonstrating the specificity of the primers of kit 3 of the present invention to the H5N1 subtype compared to other influenza A subtypes. Lane 1 is a marker, lane 2 H5N1 (−1) A/chicken/Vietnam/8/2004 (HPAI), lane 3 H5N3. The primer that amplifies a region comprising SEQ ID NO:7 of NA may be approximately 300 bp while the primer that amplifies a region comprising SEQ ID NO:1 of HA may be approximately 195 bp.

### DETAILED DESCRIPTION OF THE INVENTION

Some terms used in the present description are defined hereunder for the purpose of clarity. Well-known general molecular biology methods and techniques in the art not specifically described may be found in text books such as Sambrook et al. (ed.), Molecular Cloning: A Laboratory Manual, 3rd ed., vol. 1-3, Cold Spring Harbor Laboratory Press, Cold Spring Harbor, N.Y., 2001. This book is also available as an online reference at http://www.molecularcloning.com/.

### DEFINITIONS

Nucleotide: Includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide analog refers to moieties that function similarly to oligonucleotides but have non-naturally occurring portions. For example, oligonucleotide analogs may contain non-naturally occurring portions, such as altered sugar moieties or inter-sugar linkages, such as a phosphorothioate oligodeoxynucleotide. Functional analogs of naturally occurring polynucleotides may bind to RNA or DNA, and include peptide nucleic acid (PNA) molecules. Polynucleotides are also called polynucleic acids and these two terms are used interchangeably. The phrase “polynucleic acid” refers to RNA or DNA, as well as mRNA and cDNA corresponding to or complementary to the RNA or DNA. According to the present invention, the term “polynucleotide also encompasses peptide nucleic acids (PNA). The term “gene” comprises both sense and antisense strands of a polynucleic acid which encodes a peptide, prepeptide, protein or marker, or to a vector or plasmid containing such a polynucleic acid, although usually, only the sequence of the sense strand is given. A fragment of a polynucleotide is a shortened length of the polynucleotide.

Nucleotide sequence ambiguity: In nucleotide sequences, a few nucleotides may change or mutate over time. In such changes or mutations, the original nucleotide is replaced or substituted by another, particularly one purine for another purine, or one pyrimidine for another pyrimidine. However, one purine may also substitute for a pyrimidine and vice versa. Where a nucleotide position is ambiguous and may be represented by one or more nucleotides, standardized symbols or letters, well known to a person skilled in the art, as given in the sequence listing of this application are used. Such symbols or letters (in either upper or lower case), proposed by the International Union of Pure and Applied Chemistry (IUPAC; Cornish-Bowden (1985) Nucl. Acids Res. 13: 3021-3030) which also corresponds to WIPO Standard ST.25 Appendix 2 Table 1, are as follows:

### IUPAC Nucleotide Ambiguity Codes

### Symbol Meaning Nucleic Acid

### A A Adenine

### C C Cytosine

### G G Guanine

### T T Thymine

### U U Uracil

### M A or C

### R A or G

### W A or T

### S C or G

### Y C or T

### K G or T

### V A or C or G

### H A or C or T

### D A or G or T

### B C or G or T

### X G or A or T or C

### N G or A or T or C

Primers: Short nucleic acids, for example, DNA oligonucleotides of two or more nucleotides or more in length, for example, 6, 98 or 10 nucleotides in length, which are annealed to a complementary target DNA strand by nucleic acid hybridization to form a hybrid between the primer and the target DNA strand, then extended along the target DNA strand by a DNA polymerase enzyme. Primer pairs may be used for amplification of a nucleic acid sequence, e.g., by the polymerase chain reaction (PCR) or other nucleic-acid amplification methods known in the art.

Probes and/or primers as used herein may, for example, include at least 10 nucleotides of the nucleic acid sequences that are shown to encode specific proteins. However, probes and/or primers may also be of less than 10 nucleotides.

Polynucleotides, polynucleic acids, probes and/or primers possess a certain sequence. Sequences of interest are listed according to the present invention. Two lengths of polynucleotides, polynucleic acids, probes or primers are said to possess the same sequence when they have the same sequence. However, according to the present invention, two sequences are also said to be the same if a probe or primer can bind to both sequences.

When referring to a probe and/or primer, the term specific for (a target sequence) indicates that the probe and/or primer hybridizes under stringent conditions substantially only to the target sequence in a given sample comprising the target sequence.

Hybridization: The process wherein oligonucleotides and/or their analogs bind by hydrogen bonding, which includes Watson-Crick, Hoogsteen or reversed Hoogsteen hydrogen bonding, between complementary bases. Generally, nucleic acid consists of nitrogenous bases that are either pyrimidines (Cytosine (C), uracil (U), and thymine (T) or purines (adenine (A) and guanine (G)). These nitrogenous bases form hydrogen bonds consisting of a pyrimidine bonded to a purine, and the bonding of the pyrimidine to the purine is referred to as “base pairing.” More specifically, A will bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms which indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions in which specific binding is desired, for example, under physiological conditions in the case of in vivo assays. Such binding is referred to as “specific hybridization.” Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ concentration) of the hybridization buffer will determine the stringency of hybridization.

A person skilled in the art will appreciate that, depending on the context, the terms “binding”, “hybridizing” or “hybridization” may be used interchangeably without giving rise to ambiguity.

Accordingly, at least one primer and/or probe according to the present invention may be used to specifically hybridize to a influenza A virus nucleic acid. In particular, at least one primer and/or probe according to the present invention may be used to specifically hybridize to a H5N1 subtype of influenza A virus nucleic acid, either an RNA or DNA, and not to cross-hybridize to other subtypes of influenza A nucleic acids or to nucleic acids of other viruses. Thus, a primer or probe consists essentially of a nucleotide sequence if it includes that sequence and additional nucleotides that do not impair the ability of the primer or probe to specifically hybridize to a H5N1 virus nucleic acid under the conditions selected for performing a diagnostic assay according to the invention.

In vitro amplification: Techniques that increase the number of copies of a nucleic acid molecule in a sample or specimen. An example of amplification is the polymerase chain reaction, in which a biological sample collected from a subject is contacted with a pair of oligonucleotide primers (primer pair), under conditions that allow for the hybridization of the primers to nucleic acid template in the sample. The primer pair may be defined as to comprise an “upper” or “forward” primer and a “lower” or “reverse” primer. Each primer of the pair hydridizes to a (sense or antisense) strand of the DNA template sequence to be amplified. The primers are extended under suitable conditions, dissociated from the template, and then re-annealed, extended, and dissociated to amplify the number of copies of the nucleic acid. The product of in vitro amplification may be characterized by electrophoresis, restriction endonuclease cleavage patterns, oligonucleotide hybridization or ligation, and/or nucleic acid sequencing, using standard techniques.

Amplicon: The product of an in vitro nucleic acid amplification process is called an amplicon. The length of the amplicon may be derived by the start positions of the upper and lower primers, relative to a fixed reference of nucleotide position, usually to that of the upper (sense or coding) strand of the sequence to be amplified. For example, relative to the sense or coding strand, a theoretical 20-bp forward primer begins at position 100 and ends at position 120 and a 20-bp reverse primer starts at position 1000 and ends at position 980 (relative to the sense or coding strand). This primer pair will amplify a 900-bp amplicon from position 100 to 1000. The amplicon includes the forward and reverse primers. The length of an amplicon may also provide confirmation of successful hybridization of the primer sequences of the present invention.

Restriction site: A restriction site is a specific nucleic acid sequence recognized and cleaved by a restriction enzyme. An internal restriction site is a restriction site located within a particular nucleic acid sequence of interest.

Label: A chemical, moiety or molecule that allows detection of the label together with any molecule, surface or material to which the label is applied, attached, coupled, hybridized or bound to. Examples of labels include dyes, radiolabels, fluorescent labels, magnetic labels and enzymatic labels. Labels may be used to indicate the presence of the molecule, surface or material the label are applied, attached, coupled, hybridized or bound to. Such labels may also be called reporter labels or reporter molecules.

Biological sample: A sample of any tissue or fluid from a human, animal or plant.

### DESCRIPTION

The present invention provides oligonucleotides, method(s) and kit(s) for determining the presence of an influenza type and/or subtype virus in a biological sample or from biological material isolated and/or purified from a biological sample.

The present invention provides a method of determining the presence or absence of a type or subtype of Influenza A virus in a biological sample. This may be done by first determining the presence of a gene sequence common in Influenza A subtypes and/or the presence of one or more gene sequences present in specific subtypes.

The method of the present invention lie generally in the use of nucleic acid sequences or oligonucleotides recognizing sequences of the Influenza A virus to provide sensitive means of detection. The determining may be done, for example, by polymerase chain reaction (PCR) and/or suspension array techniques. However, any other suitable techniques of detection may also be used.

### Oligonucleotides

The present invention provides oligonucleotides for use as probes and/or primers for detecting the Influenza virus. The probes/primers of the present invention are designed to provide recognition of sequences in the Influenza A and its H5N1 subtype while allowing for single base mutations at specific locations.

The probes or primers of the invention may range from 9 to 50 nucleotides long. In particular, they may range from 12 to 30 nucleotides long. More in particularly, they may range from 15 to 25 nucleotides long. The sequences of the probes and primers used under the present invention are given below.

For example, the present method and kits may utilize specific oligonucleotides (probes and/or primer pairs) designed around the highly pathogenic region of HA segment 4 of H5N1.

According to the present invention, at least one primer pair detects the M gene (coding for the matrix protein) conserved in all influenza A strains which acts as confirmation for influenza A virus. At least one further primer pair detects the haemagglutinin (HA) segment or region of the H5N1 strain while at least another further primer pair detects the neuraminidase (NA) segment or region of the H5N1 strain. Used in this particular combination, these primer pairs produce a signature agarose gel pattern that acts as a combined internal and external confirmation for the correct strain of influenza A subtype H5N1.

The Influenza A virus encodes 8 open reading frames (ORFs), including the PB1 (segment 1), PB2 (segment 2), PA (segment 3), HA (segment 4), NP (segment 5), NA (segment 6), M (segment 7) and NS (segment 8).

In the present invention, SEQ ID NO:16, SEQ ID NO:17, SEQ ID NO:18 and/or SEQ ID NO:19 are directed to the HA region of the virus.

A person skilled in the art will appreciate that the probes or primers may further comprise at least one label.

The present invention provides oligonucleotides (for example, probes and/or primers) and nucleic acids that are labeled with suitable labels and/or reporter molecules. As such, the probes may also comprise other molecules for the detection method selected, to detect hybridized probes and target sequences. For example, such molecules may be biotin, avidin and/or strepta-avidin. Such molecules allow recognition or binding of a label or reporter molecule to a nucleic acid sequence of interest.

Accordingly, the present invention provides an isolated oligonucleotide comprising at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragment(s) thereof, derivative(s) thereof, mutation(s) thereof, and complementary sequence(s) thereof. In particular, the present invention provides an isolated oligonucleotide comprising essentially of at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragment(s) thereof, derivative(s) thereof, mutation(s) thereof, and complementary sequence(s) thereof. More particularly, the present invention provides an isolated oligonucleotide consisting of at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragment(s) thereof, derivative(s) thereof, mutation(s) thereof and complementary sequence(s) thereof, and complementary sequence(s) thereof. The isolated oligonucleotide may comprise a nucleotide sequence selected from the group consisting of SEQ ID NOS:30-60.

According to a particular embodiment, there are provided one or more specific primer pairs which are capable of detecting influenza A virus with H5N1 subtype using the PCR assay. In particular, the one step RT-PCR assay. In particular, there are provided specific primer pairs which are capable of detecting influenza A with H5N1 subtype with confirmation with one step RT-PCR assay. For example, the present method and kits may utilize specific primer pairs designed around the highly pathogenic region of HA segment 4 of H5N1. In particular, the probe, primer or primer pair may comprise at least one nucleotide sequence which hybridizes or is complementary to SEQ ID NO:1 or a fragment thereof.

The SEQ ID NO:1 or fragment thereof may comprise an internal confirmation site capable of being digested with a restriction enzyme. Any suitable restriction enzyme may be used. For example, the restriction enzyme is Mob II. However, other suitable restriction enzymes will be evident to any skilled person in the art. A restriction enzyme site within the detection region allows an additional level of confirmation that ensures that any nucleic acid sequence in biological sample or a nucleic acid sequence that has been extracted, purified and/or amplified from the biological sample that hybridizes to SEQ ID NO:1, is actually from the highly pathogenic region of the H5N1 subtype of influenza A virus. This reduces the chance of a false positive result from similar influenza A subtypes. Any suitable restriction enzyme site within the detection region can provide a source of internal confirmation for identification of the H5N1 subtype.

The method of the present invention provides a sensitive and specific detection of the highly pathogenic region of the HA segment 4 of H5N1 subtype of influenza A virus. If the detection is directed towards the highly pathogenic region and if the sequence of the highly pathogenic region mutates, it is likely that the pathogen's virulence will also be changed. The highly pathogenic region of the HA segment 4 (SEQ ID NO: 1) of H5N1 subtype of Influenza A virus appears to be unique to the H5N1 subtype. The HA segment may distinguish the N5N1 subtype from other influenza A subtypes that include the H5N3 or H5N2 subtypes. The HA segment or region may thus be used as a detection region for the H5N1 subtype.

A restriction enzyme site within the detection region allows an additional level of confirmation that ensures that any nucleic acid sequence in biological sample or a nucleic acid sequence that has been extracted, purified and/or amplified from the biological sample that hybridizes to SEQ ID NO:1, is actually from the highly pathogenic region of the HA segment 4 of H5N1 subtype of influenza A virus. This reduces the chance of a false positive result from similar influenza A subtypes. Any suitable restriction enzyme site within the detection region may provide a source of internal confirmation for identification of the H5N1 subtype.

According to the invention, there is also provided a primer comprising a nucleotide sequence set forth in SEQ ID NO:2 or SEQ ID NO:3.

There is also provided a primer comprising a nucleotide sequence set forth in SEQ ID NO:4 or SEQ ID NO:5.

Primer pair according to the invention may comprise nucleotide sequences set forth in SEQ ID NOS:2 and 3. In particular, the primer pair set forth in SEQ ID NOS: 2 and 3 amplify SEQ ID NO:6

The probe according to the invention may be a probe comprising a nucleotide sequence which hybridizes or is complementary to the sequence set forth in SEQ ID NO: 1 or to a fragment thereof.

The primer pair may comprise nucleotide sequences set forth in SEQ ID NOS: 4 and 5. In particular, the primer pair set forth in SEQ ID NOS: 2 and 3 or SEQ ID NOS: 4 and 5 amplify a sequence which hybridizes or is complementary to a sequence comprising SEQ ID NO:1 or to a fragment thereof.

The method further comprises detection of a NA segment 6 of H5N1 by contacting a further nucleic acid probe or primer which hybridizes specifically to a section of SEQ ID NO: 7 (for example, to a section at least 15 nucleotides of SEQ ID NO:7) with a nucleic acid sequence in the biological sample or contacting the further probe or primer with a nucleic acid extracted, purified or amplified from the biological sample, for a time and under conditions sufficient for specific hybridization to occur. In particular, the hybridization occur for a time and under conditions sufficient for specific hybridization to occur between the further probe or primer and the nucleic acid or sample or wherein the further probe or primer consists of a nucleotide sequence complementary to the section of SEQ ID NO:7. In particular, the further primer according to the invention may comprise a nucleotide sequence set forth in SEQ ID NO: 8 or SEQ ID NO: 9. The further primer pair may comprise nucleotide sequences set forth in SEQ ID NOS: 8 and 9. Particularly, the further primer pair set forth in SEQ ID NOS: 8 and 9 amplify SEQ ID NO: 7. In a preferred embodiment the further probe comprises a section of at least 15 nucleotides set forth in SEQ ID NO: 7. More particularly, the forward primer of SEQ ID NO:8 and the reverse primers of either SEQ ID NOS51 or 52 amplify SEQ ID NO:7 and a probe comprising the sequence of SEQ ID NO:60 is capable of binding to the amplicon.

Detection of the neuraminidase (NA) segment 6 of H5N1 subtype of Influenza A virus provides further confirmation that it is actually the H5N1 subtype that has been detected in a biological sample or nucleic acid extracted, purified or amplified from the biological sample that hybridizes to SEQ ID NO 1 from the highly pathogenic region of the haemagglutinin (HA) segment 4 of H5N1 subtype of Influenza A virus. This further reduces the chance of a false positive. Detection of SEQ ID NO: 7 in a sample provides a source of external confirmation.

According to another aspect, there is also provided a method of determining the presence or absence of a type or subtype of Influenza A virus in a biological sample, the method comprising the steps of: providing a biological sample; contacting at least a nucleic acid probe, primer or primer pair with the biological sample or contacting the probe, primer or primer pair with a nucleic acid extracted, purified and/or amplified from the biological sample, wherein the probe, primer or primer pair hybridizes to a section of SEQ ID NO:10 (for example, to a section at least 15 nucleotides of SEQ ID NO:10) or a fragment thereof; and detecting the hybridization resulting from the contacting step, wherein the matrix segment of a type or subtype of Influenza A virus is determined in a biological sample when hybridization of the probe, primer or primer pair to the sample or to a nucleic acid extracted, purified and/or amplified from said biological sample is detected. In a preferred embodiment the method further comprises detection of the matrix segment of all influenza A virus subtypes by contacting another nucleic acid probe, primer or primer pair which hybridizes specifically to a section of SEQ ID NO: 10 (for example, to a section at least 15 nucleotides of SEQ ID NO:10) with the biological sample or contacting the other probe, primer or primer pair with a nucleic acid extracted, purified and/or amplified from the biological sample, for a time and under conditions sufficient for specific hybridization to occur between the other probe or primer and the nucleic acid or sample or wherein the other probe, primer or primer pair consists of a nucleotide sequence complementary to the section of SEQ ID NO: 10.

Detection of the matrix segment 7, conserved across and specific to all subtype of Influenza A virus may provide further confirmation that a positive result that has been detected in a biological sample or nucleic acid extracted, purified and/or amplified from the biological sample that hybridizes to SEQ ID NO 1 from the highly pathogenic region of the haemagglutinin (HA) segment 4 or SEQ ID 7 of the neuraminidase (NA) segment 6 is actually from an Influenza A virus. This further reduces the chance of a false positive. Detection of SEQ ID NO: 10 in a sample provides a further source of external confirmation.

The primer comprises a nucleotide sequence set forth in SEQ ID NO: 11 or SEQ ID NO: 12. The other primer pair comprises nucleotide sequences set forth in SEQ ID NOS: 11 and 12. In particular, the other primer pair set forth in SEQ ID NOS: 11 and 12 amplify SEQ ID NO: 10. According to another aspect, the probe comprises a section of 15 nucleotides set forth in SEQ ID NO: 10.

According to another aspect, the method further comprises detection of a NA segment 6 of H5N2 by contacting a further nucleic acid probe, primer or primer pair which hybridizes specifically to a section of SEQ ID NO: 13 (for example, to a section at least 15 nucleotides of SEQ ID NO:13) with the biological sample or contacting the further probe, primer or primer pair with a nucleic acid extracted, purified and/or amplified from the biological sample, for a time and under conditions sufficient for specific hybridization to occur between the further probe or primer and the nucleic acid or sample or wherein the further probe, primer or primer pair comprises a nucleotide sequence complementary to the section of SEQ ID NO:13.

Detection of the neuraminidase (NA) segment 6 of H5N2 subtype of Influenza A virus provides negative confirmation that it is not the H5N1 subtype that has been detected in a biological sample or nucleic acid extracted, purified and/or amplified from the biological sample. Samples that hybridize to SEQ ID NO 13 from the neuraminidase (NA) segment 6 of H5N2 subtype of Influenza A virus and not the SEQ ID No 1 or 7 of the N5H1. This further reduces the chance of a false positive. Detection of SEQ ID NO:13 in a sample provides a further source of external negative confirmation.

The further primer may comprise a nucleotide sequence set forth in SEQ ID NO: 14 or SEQ ID NO: 15. In particular, the further primer pair comprises nucleotide sequences set forth in SEQ ID NOS: 14 and 15. The further primer pair set forth in SEQ ID NOS: 14 and 15 amplify SEQ ID NO: 13. According to another aspect, the further probe comprises a section of 15 nucleotides set forth in SEQ ID NO: 13.

According to another aspect, the detection of presence or absence of a H5N1 subtype of Influenza A virus using one of more probed, primer or primer pair selected form the group consisting of SEQ ID NOS: 1, 2, 3, 4, 5, 6, 7, 8, and 9 or a complementary sequence thereof, is analyzed by chromatography.

The present invention provides a simple, sensitive and/or specific diagnostic test. By use of the probes and/or primers described herein, the method(s) and/or kit(s) are made more sensitive and specific than the detection methods of the prior art. Such tests may be by suspension array technology methods. Alternatively, such tests may be by a one step PCR method or a two step PCR. Any other suitable techniques may also be used.

Thus, in one embodiment, the H5N1 detection method and kit of the present invention lies generally in use of a set of probes that are specific for the M gene as well as the highly pathogenic region of the HA gene and the NA gene of H5N1 subtype of influenza A virus genome for detection of presence of the virus.

The method may be performed, for example by detecting viral nucleic acids sequences present in the sample using one or more probes recognizing and hybridizing to the M gene of the Influenza A virus. Further probes may be used to recognize and hybridize to the HA gene from nucleotide 999 to nucleotide 1091, or using one or more probes recognizing and hybridizing to the region of the HA gene from nucleotide 943 to nucleotide 1114 as well as to the NA gene.

### Detection by Suspension Array Technology

According to the present invention, the sample nucleic acids may optionally be amplified, for example, by PCR, before being detected by suspension array technology. When used in suspension array techniques, one or more of the sequences of the present invention are bound to a support such as particles or microbeads. According to one embodiment, each probe is used to label an unique microbead identifiable by a specific ratio of two dyes that give a unique fluorescent signal when excited by a light of a specific wavelength. The microbeads with bound or hybridized nucleic acids from the sample are then distinguished by being sorted or separated from microbeads without bound or hybrized nucleic acids. The microbeads with bound or hybridized nucleic acids are then identified by a first laser light based on the two dyes that fluoresces at a specific intensity. A second laser light quantitates the amount of probe bound or hybridized with nucleic acids from the a sample by causing a label bound to either the probe or nucleic acid to fluoresce.

Suspension array technology, is exemplified by U.S. Pat. No. 6,939,720, U.S. Pat. No. 6,916,661, U.S. Pat. No. 6,939,720 or U.S. Pat. No. 6,514,295, assigned to the Luminex Corporation (the whole contents of which are herein incorporated by reference).

According to this technology, a liquid suspension array of up to 100 sets of 5.6 micron microbeads, each stained or dyed with different ratios of two spectrally distinct fluorophores permitting each of the 100 sets of microbeads to be distinguished. Each set of microbeads may be conjugated with a different capture molecule such as the probes of the present invention. The conjugated microbeads may then be mixed and incubated with samples in a micro plate well to allow hybridizing of nucleic acids (for example, RNA purified from the sample, cDNA converted from the sample or DNA amplicons amplified by PCR) in the sample with the probes conjugated to the microbeads. Unbound nucleic acids may be separated for example, by centrifugation. The nucleic acids in the sample may also be prelabelled with biotin, for example. The biotin label allows for binding to another fluorescent reporter molecule, for example, streptavidin-phycoerythrin.

Following incubation with fluorescently labeled reporter molecules, the contents of each micro plate well are analysed by being drawn up into a flow cytometer where the microbeads are aligned in single file through a flow cell. Two lasers excite the microbeads individually. A first (red) classification laser excites the dyes in each microbead to give off fluorescent signals, the intensities of the fluorescent signals identifying each microbead's spectral address or identity. A second (green) reporter laser excites the reporter molecule associated with the microbead or sample, which allows quantitation of the captured sample nucleic acids. The fluorescent signals are then simultaneously recorded by for each microbead, translating the signals into data for each bead-based assay. This analysis step also distinguishes microbeads with captured sample nucleic acids from the beads without captured sample nucleic acids.

Accordingly, the present invention provides detecting of binding of the oligonucleotide(s) and the sample and/or nucleic acid(s) by suspension array technology. The suspension array technology may the steps of:

(a) providing a biological sample;
(b) contacting at least one oligonucleotide with at least one nucleic acid in the biological sample, or contacting the oligonucleotide with at least one nucleic acid extracted, purified and/or amplified from the biological sample, wherein the oligonucleotide comprises at least one nucleotide sequence selected from the group consisting of:
SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, fragments(s) thereof, derivatives(s) thereof, mutation(s) thereof; and a complementary sequence(s) thereof;
and
(c) detecting any binding resulting from the contacting in step (b), whereby an H5N1 subtype of an Influenza A virus is present when binding is detected.

Accordingly, the method of the present invention may comprise:

(i) providing a biological sample;
(ii) labeling the at least one nucleic acid in the biological sample or extracted, purified or amplified from the sample with at least one reporter label;
(iii) immobilizing at least one probe to at least one microbead comprising at least one fluorescent dye;
(iv) contacting the at least one probe with the at least one nucleic acid to allow binding of the probe(s) and nucleic acid(s);
(v) identifying microbeads based on the fluorescent intensity of the at least one fluorescent dye with a first laser light and detecting binding of nucleic acid(s) to probe(s) immobilized on identified microbead(s) with a second laser light based on the reporter label(s);
whereby the detection of binding of the nucleic acid(s) to probe(s) indicates the presence of the H5N1 subtype of Influenza A virus.

The method of the invention may comprise detecting binding or hybridization by an amplification step. For example, the method may comprise detection means comprising a polymerase chain reaction (PCR) format using one or more probe or primer or primer pairs.

### Detection by Polymerase Chain Reaction

A person skilled in the art will appreciate that the probes of the present invention may also be used as a primers for PCR detection as they demarcate a stretch of nucleic acid that may be amplified. Accordingly, the terms primers and probes may be referred to interchangeably depending on the context or detection method wherein they are used. Real-time PCR detection under in the present invention may be performed using PCR platforms such as the Roche LightCycler™, the Stratagene Real-time PCR system, the Applied Biosystems ABI 7000 real time PCR analyzer or any other suitable detection platform.

The method may be performed, for example, with PCR by amplifying nucleic acids present in the sample using a forward primer and a reverse primer pair selective for the region of the Influenza B genome such as the Matrix gene to obtain an amplification product or amplicon. Any binding, hybridization and/or amplification product may then detected. The amplification product may be detected, for example, by determining the length of the amplification product in nucleotides, either by a chromatographic method or by a gel electrophoretic method, e.g by electrophoresis in 2 or 3% agarose, run in conjunction with a molecular weight marker set of suitable resolution. The presence of an amplification product having a length in nucleotides that is the sum of the forward primer length, the reverse primer length and the separation length indicates the presence of the Matrix nucleic acid of the Influenza B virus in the sample.

Alternatively, the product may be detected with PCR using a hybridization probe, for example using real-time fluorescent detection such as the Taqman™ system (Applied Biosystems, Foster City, Calif.) wherein a fluorescent label attached to the probe is released by the polymerase when the probe is bound or hybridized to the target sequence and extended by the polymerase. The amount of label released thus gives an indication of the quantity of target sequences present in the sample.

When PCR methods are used to detect hybridization, the hybridization probe may comprise a nucleotide sequence that is the same as that of a portion of the amplification product that would be obtained using the amplification primers selected and a Influenza B virus genomic nucleic acid as a template.

When used in PCR, a pair of sequences of the present invention may function as the forward (or upper) primer and lower (or reverse) primers for a stretch of Influenza B sequence. These primers then allow the stretch of Influenza B sequence to be amplified by a PCR method. The amplicon thus obtained may be identified by its size (length or molecular weight) from gel electrophoresis and/or from a probe binding to the amplicon.

According to the present invention, the at least one oligonucleotide may be two oligonucleotides forming a primer pair and the step (c) of detecting may be by a polymerase chain reaction.

As an example, to detect the presence of the H5N1 subtype of Influenza A, the HA-114 amplicon may be amplified. The primer pair may bind to the nucleic acid(s) and amplify at least one amplicon comprising the sequence of SEQ ID NO:6. The primer pair may comprise at least one forward primer comprising the nucleotide sequence SEQ ID NO:25 and at least one reverse primer comprising the nucleotide sequence SEQ ID NO:26. In particular, the forward primer may comprise at least the nucleotide sequence of SEQ ID NO:34 and the reverse primer comprises at least the one nucleotide sequence selected from the group consisting of SEQ ID NO:35 and SEQ ID NO:36.

As another example, the HA-195 amplicon may be amplified. The primer pair may bind to the nucleic acid(s) and amplify at least one amplicon comprising the sequence of SEQ ID NO:7. The primer pair may comprise at least one forward primer comprising the nucleotide sequence SEQ ID NO:18 and at least one reverse primer comprising the nucleotide sequence SEQ ID NO:19. In particular, the forward primer may comprise at least one nucleotide sequence selected from the group consisting of SEQ ID NOS:37 to 44 and the reverse primer may comprise at least one nucleotide sequence selected from the group consisting of SEQ ID NOS:45 and SEQ ID NO:48. A probe comprising at least one sequence selected from the group consisting of SEQ ID NOS:49 and 50 may bind to the amplicon.

The step of detecting may be by detection of at least one label released by the polymerase chain reaction, determination of the molecular weight of at least one amplicon obtained from the polymerase chain reaction and/or the detecting may be by detection of binding of at least one probe to the at least one amplicon. The polymerase chain reaction may be followed by electrophoresis.

For detection by PCR, step (b) of contacting and/or the step (c) of binding, and/or the binding of a probe to an amplicon, is for a time and under conditions sufficient for specific contacting or binding to occur between the oligonucleotide and sample or nucleic acid(s) and/or the probe to the amplicon.

The biological sample may be from a human or non-human animal suspected to be infected with the Influenza A H5N1 virus. Further, the oligonucleotide and/or the sample or nucleic acid may be labeled.

### Kits

The present invention also generally relates to a kit for determining the presence of an influenza virus in a biological sample or from biological material isolated and/or purified from a biological sample.

The present invention also provides a kit comprising at least one nucleic acid, the nucleic acid selected from the group consisting of: SEQ ID NO:8, SEQ ID NO:9, SEQ ID NO:16, SEQ ID NO:17, SEQ ID NO:18, SEQ ID NO:19, SEQ ID NO:20, SEQ ID NO:21, SEQ ID NO:22, and or a fragment thereof.

In particular, the kit may be for the detecting the presence or absence of an Influenza A virus. More in particular, the kit may be for the detecting the presence or absence of an Influenza A subtype H5N1 virus. The kit may be for use with a biological sample, The sample may be from a human or animal which can be infected with a H5N1 subtype of Influenza A virus.

The kit may further comprise information for use of the kit. It may be for example an illustrative information provided by the manufacturer. In particular, the present invention provides a simple RT-PCR detection kit. Such a kit comprises one or more primers and/or probes according to the invention, for example a kit may contain primers consisting of one or more polynucleotides comprising a nucleotide sequence of SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29, and or a fragment thereof. The kit for detection by suspension array technology may comprise at least one microbead, at least one fluorescent dye, at least one reporter label and/or at least one probe.

A kit according to the invention may optionally include a positive control nucleic acid, for example a nucleic acid, or at least a portion thereof, comprising the HA, NA and or M region of H5N1 influenza A virus, as either RNA or DNA.

A person skilled in the art will appreciate that the probes and/or primers may further comprise at least one label. A label may be a chemical, moiety or molecule that allows detection of the label together with any molecule, surface or material to which the label is applied, attached, coupled, hybridized or bound to. Examples of labels include dyes, radiolabels, fluorescent labels, magnetic labels and enzymatic labels.

In addition, the probes may also comprise other molecules for the detection method selected to detect hybridized probes and target sequences. Examples of such molecules may be biotin, avidin and/or strepta-avidin. Such molecules allow recognition or binding of a label or reporter molecule to a sequence of interest.

Having now generally described the invention, the same will be more readily understood through reference to the following examples which are provided by way of illustration, and are not intended to be limiting of the present invention.

### EXAMPLES

### Example 1

### Materials and Methods

Standard molecular biology techniques known in the art and not specifically described were generally followed as described in Sambrook and Russel, Molecular Cloning: A Laboratory Manual, Cold Springs Harbor Laboratory, New York (2001).

### Standards

A 10-fold dilution of the stock virus was prepared in serum obtained from a healthy volunteer. RNA was extracted using the QIAGEN Viral RNA Kit (QIAGEN GMbH, Germany).

### Patient Specimens

Virus isolation was performed on a serum specimen of avian flu cases. RNA was directly extracted from the specimen using a Qiagen QIAamp viral RNA extraction kit (catalog no. 52906) according to the instructions given in the product insert. RNA was also extracted from infected birds in a manner understood by those in the art and treated with Qiagen RLT buffer, a proprietary product that contains guanidine and β-mercaptoethanol.

The isolated samples included:

### H5N1 (−1) A/chicken/Vietnam/8/2004 (HPAI),

H5N3 (−1) A tern/Australia/75/(LPAI),

### H7N3 (−1) A/chicken/queensland/1994 (HPAI) and

### H7N7 (−1) A/duck/Victoria/1976 (LPAI).

### Other Viruses

RNA was directly extracted from the stock vial obtained from the American Type Culture Collection (ATCC; VA, USA) using the QIAGEN Viral RNA Mini Kit (QIAGEN GMbH, Germany) according to the instructions given in the product insert. It would be understood by those in the art that RNA isolated from any non influenza virus would be sufficient to provide a suitable negative control.

### MRC-5 Cell Line

Total RNA was extracted directly from the normal diploid human fibroblast cell line MRC-5 (ATCC CCL171) using a Qiagen RNA extraction kit (catalog no. 74104) and RNA is quantitated using a spectrophotometer.

### Detection Using the Primer Pairs

RNA was extracted from samples thought to contain influenza A subtype H5N1 RNA as assessed by known methods. The RNA was then converted to DNA using a reverse transcriptase or any other method known in the art. A sample mixture was converted into cDNA in a typical manner using a 1st Strand cDNA Synthesis Kit for RT-PCR (sold by Roche, Basel, Switzerland, catalog no. 1 483 188).

### Suspension Array

Suspension array apparatus and microbeads were from Luminex Corporation and Alexa488 fluor dye was from Invitrogen/Molecular Probes (Eugene, Oreg.). The machine used was the Luminex 100 set from Luminex Corporation (Austin, Tex.).

### Example 2

### Influenza B Virus Detection by Suspension Array Technology

Probes While the following probes of the present invention are designed to detect influenza A virus and further determine subtype H5N1 specifically, by suspension array technology, a person skilled in the art will appreciate that the following sequences may also be used in other suitable detection methods for influenza A virus subtype H5N1 as well, such as in situ hybridization, nuclease protection assay, etc.

### Probes for M Gene

The following probes were designed as generic probes to detect all influenza A type by recognizing and/or demarcating a portion of the M gene (Matrix gene segment 7) from nucleotide 28 to nucleotide 171.

### MP-168 (Upper)

### 5′-GAGNCTTCTAACCGAGGTCGAAAC-3′ (SEQ ID NO:23)

### N=A, T, C or G

In particular, the sequence is

### 5′-GAGYCTTCTAACCGAGGTCGAAAC-3′ (SEQ ID NO:21)

### Y=C/T

Accordingly, the particular variations for MP-168 (Upper) are:

### 5′-GAGCCTTCTAACCGAGGTCGAAAC-3′ (SEQ ID NO:30)

### 5′-GAGTCTTCTAACCGAGGTCGAAAC-3′ (SEQ ID NO:31)

### MP-168 (Lower)

### 5′-TTAGTCAGAGGTGACAGNATTGGTC-3′ (SEQ ID NO:24)

### N=A, T, C or G

In particular, the sequence is

### 5′-TTAGTCAGAGGTGACAGRATTGGTC-3′ (SEQ ID NO:22)

### R=A/G

Accordingly, the particular variations for MP-168 (Lower) are:

### 5′-TTAGTCAGAGGTGACAGMTTGGTC-3′ (SEQ ID NO:32)

### 5′-TTAGTCAGAGGTGACAGGATTGGTC-3′ (SEQ ID NO:33)

### MP-168 (Probe)

### 5′-GCTTTGAGGGGGCCTGANGGN-3′ (SEQ ID NO:53).

### N=A, T, C or G

In particular, the sequence is:

### 5′-GCTTTGAGGGGGCCTGAYGGR-3′ (SEQ ID NO:54)

### Y=C/T,

### R=A/G

Accordingly, the particular variations for the MP-168 probe are:

### 5′-GCTTTGAGGGGGCCTGACGGA-3′ (SEQ ID NO:56)

### 5′-GCTTTGAGGGGGCCTGATGGA-3′ (SEQ ID NO:57)

### 5′-GCTTTGAGGGGGCCTGACGGG-3′ (SEQ ID NO:58)

### 5′-GCTTTGAGGGGGCCTGATGGG-3′ (SEQ ID NO:59)

### Probes for HA Gene

The following two probes were designed to detect the HA gene, segment 4 of the H5N1 subtype of Influenza A virus by recognizing and/or demarcating portion of the HA gene from nucleotide 999 to nucleotide 1091 of the HA gene segment:

### HA-114 (Upper)

### 5′-CAAACANATTANTNCTTGCNACWG-3′ (SEQ ID NO:25)

### N=A, T, C or G

In particular, the sequence is

### 5′-CAAACARATTARTYCTTGCDACWG-3′ (SEQ ID NO:16)

### R=A/G

### Y=C/T

### D=A/G/T

### W=A/T

More particularly, the sequence is

### 5′-CAAACAGATTAGTYCTTGCGACTG-3′ (SEQ ID NO:34)

### HA-114 (Lower)

### 5′-CCTGCCATCCTCCNTCTATAAA-3′ (SEQ ID NO:26)

### N=A, T, C or G

In particular, the sequence is

### 5′-CCTGCCATCCTCCYTCTATAAA-3′ (SEQ ID NO: 17)

### Y=C/T

Accordingly, the particular variations for HA-114 (Lower) are:

### 5′-CCTGCCATCCTCCCTCTATAAA-3′ (SEQ ID NO:35)

### 5′-CCTGCCATCCTCCTTCTATAAA-3′ (SEQ ID NO:36)

The following probes were designed to detect HA gene, segment 4 of the H5N1 subtype of Influenza A virus by recognizing and/or demarcating a portion of the HA gene from nucleotide 943 to nucleotide 1114 of the HA gene segment:

### HA-195 (Upper)

### 5′-GCCATTCCACAANATNCANCC-3′ (SEQ ID NO:27)

### N=A, T, C or G

In particular, the sequence is

### 5′-GCCATTCCACAAYATMCAYCC-3′ (SEQ ID NO:18)

### Y=C/T

### M=A/C

Accordingly, the particular variations for HA-195 (Upper) are:

### 5′-GCCATTCCACAACATACACCC-3′ (SEQ ID NO:37)

### 5′-GCCATTCCACAATATACACCC-3′ (SEQ ID NO:38)

### 5′-GCCATTCCACAACATCCACCC-3′ (SEQ ID NO:39)

### 5′-GCCATTCCACAATATCCACCC-3′ (SEQ ID NO:40)

### 5′-GCCATTCCACAACATACATCC-3′ (SEQ ID NO:41)

### 5′-GCCATTCCACAATATACATCC-3′ (SEQ ID NO:42)

### 5′-GCCATTCCACAACATCCATCC-3′ (SEQ ID NO:43)

### 5′-GCCATTCCACAATATCCATCC-3′ (SEQ ID NO:44)

### HA-195 (Lower)

### 5′-TANCCATACCAACCATCTANCATT-3′ (SEQ ID NO:28)

### N=A, T, C or G

In particular, the sequence is

### 5′-TAYCCATACCAACCATCTAYCATT-3′ (SEQ ID NO:19)

### Y=C/T

Accordingly, the particular variations for HA-195 (Lower) are:

### 5′-TACCCATACCAACCATCTACCATT-3′ (SEQ ID NO:45)

### 5′-TACCCATACCAACCATCTATCATT-3′ (SEQ ID NO:46)

### 5′-TATCCATACCAACCATCTACCATT-3′ (SEQ ID NO:47)

### 5′-TATCCATACCAACCATCTATCATT-3′ (SEQ ID NO:48)

### HA-195 (Probe)

### 5′-GNCATTCCCCGATGGTGAGAGG-3′

### (SEQ ID NO:29)

### N=A, T, C or G

In particular, the sequence is

### 5′-GRCATTCCCCGATGGTGAGAGG-3′ (SEQ ID NO:20)

### R=A/G

Accordingly, the particular variations for HA-195 (Probe) are:

### 5′-GACATTCCCCGATGGTGAGAGG-3′ (SEQ ID NO:49)

### 5′-GGCATTCCCCGATGGTGAGAGG-3′ (SEQ ID NO:50)

### Probes for NA Gene in H5N1

The following probes were designed to detect the NA gene, segment 6 of the H5N1 subtype of Influenza A virus by recognizing and/or demarcating a 300 base pair portion of the NA gene from nucleotide 528 to nucleotide 805 of the NA gene segment.

### NA-300 (Upper)

### 5′-TGATGGCACCAGTTGGTTGAC-3′ (SEQ ID NO:8).

### NA-300 (Lower)

### 5′-GCATCAGGATAACAGGAGCAYTC-3′ (SEQ ID NO: 9)

### Y=C/T.

Accordingly, the particular variations for the NA-300 (Lower) primer are:

### 5′-GCATCAGGATAACAGGAGCACTC-3′ (SEQ ID NO:51)

### 5′-GCATCAGGATAACAGGAGCATTC-3′ (SEQ ID NO:52)

### NA-300 (Probe)

### 5′-ACAGCCACAGCCCCATTGTCTG-3′ (SEQ ID NO:60)

### Probes for NA Gene in H5N2 Genome as Negative Confirmation

A specific primer pair was designed to detect NA gene, segment 6 of the H5N2 subtype of Influenza A virus by amplifying a 361 base pair portion of the NA gene from nucleotide 440 to nucleotide 774 of the NA gene segment. A person skilled in the art will appreciate that the amplicon length comprises the difference in the nucleotide position (774 minus 440) plus the length of the reverse primer (27 bp) to yield an amplicon length of 361. This specific primer pair is designed to detect H5N2 subtype-NA gene as a confirmation of specificity.

### Upper Primer to NA Gene Beginning at Position 440

5′-AATGAGTTGGGTGTTCCGTTTC-3′ (SEQ ID NO:14) (22-mer)

### Lower Primer to NA Gene Ending at Position 774

5′-AACAGGAACATTCCTCTATATGCTGAG-3′ (SEQ ID NO:15) (27-mer)

### Immobilization of Probes to Microbeads

Probes were immobilized to Luminex microbeads via the microbeads' surface carboxyl groups. After immobilization, the microbeads are mixed to form a multiplexed set. For comprehensive screening of samples, all possible combinations of the probe sequences of the present invention may be included in the multiplexed set. Microbeads with suitable control sequences immobilized to them may also be included in the multiplexed set.

### Samples

Nucleic acids from both Influenza A H5N1 and H7N3 subtypes, as well as Influenza B samples were amplified by RT-PCR (protocol below in Example 3 below) using the as cDNA and an estimated copy number was then mixed with microbeads for hybridization.

### Hybridization

Samples are then contacted with a set of multiplexed microbeads individually containing the HA-195 probe variations (SEQ ID NOS:49 and 50) in approximately equal abundance, MP-168 probe variations (SEQ ID NOS 56-59) in approximately equal abundance, and the NA-300 probe (SEQ ID NO:60). Any hybridization allowed to occur in a hybridization buffer comprising 2.25 mol/L tetramethyl ammonium, 0.75 g/L sodium docecyl sulfate, 37.5 mmol/L Tris (pH8.0) and 1.5 nmol/L EDTA (pH8.0 for one hour at 37° C. After hybridization, 2 μL of a 10-g/L solution of streptavidin-phycoerythrin was added and incubated at room temperature for 30 min.

The mixture was then diluted with 200 μL of the hybridization buffer and analyzed with a Luminex 100 system machine to detect and quantify presence of target nucleic acid sequences in the sample.

### Results

The results of the suspension array detection assay are shown in Tables 1 and 2, as fluorescent intensity (no units of measurement) as obtained by the machine. For both tables, an intensity of 100 was selected as an arbitrary threshold cut-off.

Primer variants used in the amplification step were

FluA, NA 300, specific to H5N1 subtype (SEQ ID NOS:8 and 9)
FluA, HA 195, specific to H5N1 subtype (SEQ ID NOS:37 and 45)
FluA, Matrix 168, specific to any Influenza A (SEQ ID NOS:31 and 33)

For both tables, it can be seen that despite high copy numbers of non-target nucleic acids present, the probe of the present invention was able to detect low copy numbers of H5N1 nucleic acids, showing the specificity of the probes of the invention.

TABLE 1

| | | Cut off value | | |
|---|---|---|---|---|
| | | 100 | 100 | 100 |
| | RNA | 084-Flu A-H | 085-Flu A-M | 086-Flu A-N |
| Sample | copies/rxn | Probe | Probe | Probe |
| tRNA 1 | 0 | 30 | 36 | |
| tRNA 2 | 0 | 30 | 39 | 32 |
| Flu B (−3) 1 | est > 20,000 | 37 | 35 | 36 |
| Flu B (−3) 2 | est > 20,000 | 27 | 34 | 36 |
| H5N3 (−3) 1 | est > 20,000 | 38 | 2783 | 37 |
| H5N3 (−3) 2 | est > 20,000 | 37 | 2716 | 36 |
| H7N3 (−3) 1 | est > 20,000 | 32 | 3456 | 29 |
| H7N3 (−3) 2 | est > 20,000 | 33 | 3653 | 34 |
| H7N7 (−3) 1 | est > 20,000 | 34 | 3724 | |
| H7N7 (−3) 2 | est > 20,000 | 39 | 3642 | 44 |
| H5N1 (−7) 1 | 2 | 637 | 276 | 355 |
| H5N1 (−7) 2 | 2 | 34 | 317 | 195 |
| H5N1 (−6) 1 | 20 | 1346 | 1760 | 1219 |
| H5N1 (−6) 2 | 20 | 945 | 1615 | 549 |
| H5N1 (−5) 1 | 200 | 2180 | 2937 | 2291 |
| H5N1 (−5) 2 | 200 | 2303 | 3251 | 2572 |

TABLE 2

| | | Cut off value | | |
|---|---|---|---|---|
| | | 100 | 100 | 100 |
| | RNA | 084-Flu A-H | 085-Flu A-M | 086-Flu A-N |
| Sample | copies/rxn | Probe | Probe | Probe |
| tRNA 1 | 0 | 33 | 35 | 33 |
| tRNA 2 | 0 | 28 | 29 | 33 |
| H5N1 (−7) 1 | 2 | 925 | 239 | 35 |
| H5N1 (−7) 2 | 2 | 31 | 188 | 317 |
| H5N1 (−6.5) 1 | 6 | 715 | 505 | 575 |
| H5N1 (−6.5) 2 | 6 | 848 | 258 | 201 |
| H5N1 (−6) 1 | 20 | 1476 | 1372 | 1130 |
| H5N1 (−6) 2 | 20 | 950 | 872 | 984 |
| H5N1 (−5.5) 1 | 60 | 1898 | 1937 | 2001 |
| H5N1 (−5.5) 2 | 60 | 2311 | 2201 | 2435 |
| H5N1 (−5) 1 | 200 | 3793 | 3410 | 3583 |
| H5N1 (−5) 2 | 200 | 2517 | 3192 | 2443 |
| SARS (−2) 1 | 200,000 | 33 | 99 | 29 |
| SARS (−2) 2 | 200,000 | 24 | 31 | 24 |

### Example 3

### Detection by Polymerase Chain Reaction with H5N1 and H5N2 Subtype Confirmation

These primers were designed to detect influenza A virus and further subtype H5N1 and H5N2, specifically. These primer sets were designed for gel-based RT-PCR. This means amplified products are detectable by ethidium bromide staining of amplified products after agarose gel electrophoresis. All primers described here were designed based on the sequences provided by NCBI Influenza Virus Sequence Database.

(http://www.ncbi.nlm.nih.gov/genomes/influenza/list.cgi)

### Primer Pair for M Gene for all Influenza a Subtypes

One primer pair was designed as a generic primer pair to detect all influenza A type by amplifying a 168 base pair portion of the M gene (Matrix gene segment 7) from nucleotide 28 to nucleotide 171.

A. Upper primer

The forward primer, also herein called an upper primer was a 24-mer that hybridises to the M gene beginning at position 28 and has the sequence of SEQ ID NO: 11 5′-GAGTCTTCTAACCGAGGTCGAAAC-3′

### B. Lower Primer

The reverse primer, called herein a lower primer is a 25-mer that hybridizes to the M gene ending at position 171 and has a sequence of SEQ ID NO: 12 5′-TTAGTCAGAGGTGACAGGATTGGTC-3′.

C. Amplicon Made by this Primer Set

The amplicon made by this primer set has the sequence of SEQ ID NO: 10 GAGTCTTCTAACCGAGGTCGAAACGTACGTTCTCTCTATCATCCCGTCAGGC CCCCTCAAAGCCGAGATCGCGCAGAAACTTGAAGATGTCTTTGCAGGAAAGA ACACCGATCTCGAGGCTCTCATGGAGTGGCTAAAGACAAGACCAATCCTGTC ACCTCTGACTAA (168 bp).

Primer Pair for HA Gene in H5N1

Another specific primer pair was designed to detect HA gene, segment 4 of the H5N1 subtype of Influenza A virus by amplifying a 114 base pair portion of the HA gene from nucleotide 999 to nucleotide 1091 of the HA gene segment.

### A. Upper Primer

The forward primer, also herein called an upper primer was a 24-mer that hybridises to the HA gene beginning at position 999 and has the sequence of SEQ ID NO: 2 5′-CAAACAGATTAGTCCTTGCGACTG-3′ (24 bp).

### B. Lower Primer

The reverse primer, called herein a lower primer is a 22-mer that hybridizes to the HA gene ending at position 1091 and has a sequence of SEQ ID NO:3 5′-CYTGCCATCCTCCCTCTATAAA-3′ (22 bp). In this case Y may be either C or T.

C. Amplicon Made by this Primer Set

The amplicon made by this primer set has the sequence of SEQ ID NO: 6 CAAACAGATTAGTCCTTGCGACTGGGCTCAGAAATAGCCCTCAAAGAGAGAG AAGAAGAAAAAAGAGAGGACTATTTGGAGCTATAGCAGGTTTTATAGAGGGA GGATGGCARG (114 bp).

This amplified region comprises the highly pathogenic region sequence of SEQ ID NO:1 5′-AGAAGAAGAAAAAAG-3′. Either of SEQ ID NOS:1 or 6 may be digested by a restriction enzyme Mbo II providing an internal confirmation that detection of this region of the HA gene segment 4 of the H5N1 subtype of Influenza A has occurred.

Another Primer Pair for HA Gene in H5N1

Another specific primer pair was designed to detect HA gene, segment 4 of the H5N1 subtype of Influenza A virus by amplifying a 195 base pair portion of the HA gene from nucleotide 943 to nucleotide 1114 of the HA gene segment.

### A. Upper Primer

The forward primer, also herein called an upper primer is a 21-mer that hybridises to the HA gene beginning at position 943 and has the sequence of SEQ ID NO: 4 5′-GCCATTCCACAAYATACACCC-3′ (21 bp) In this case Y may be either C or T.

### B. Lower Primer

The reverse primer, called herein a lower primer is a 24-mer that hybridizes to the HA gene ending at position 1114 and has a sequence of SEQ ID NO: 5 5′-TACCCATACCAACCATCTACCATT-3′ (24 bp).

C. Amplicon Made by this Primer Set

The amplicon made by this primer set has the sequence of SEQ ID NO:55: GCCATTCCACAACATACACCCTCTCACCATCGGGGAATGCCCCAAATATGTG AAATCAAACAGATTAGTCCTTGCGACTGGGCTCAGAAATAGCCCTCAAAGAG AGAGAAGAAGAAAAAGAGAGGACTATTTGGAGCTATAGCAGGTTTTATAGA GGGAGGATGGCAGGGAATGGTAGATGGTTGGTATGGGTA (195 bp)

This amplified region comprises the highly pathogenic region sequence of SEQ ID NO:1 5′-AGMGAAGAAAAAAG-3′. As such, either of SEQ ID NOS: 1 or 6 may be digested by a restriction enzyme Mbo II providing an internal confirmation that detection of this region of the HA gene segment 4 of the H5N1 subtype of Influenza A has occurred.

### Primer Pair for NA Gene in H5N1

Another specific primer pair was designed to detect NA gene, segment 6 of the H5N1 subtype of Influenza A virus by amplifying a 300 base pair portion of the NA gene from nucleotide 528 to nucleotide 805 of the NA gene segment.

### A. Upper Primer

The forward primer, also herein called an upper primer is a 21-mer that hybridises to the NA gene beginning at position 528 and has the sequence of SEQ ID NO: 8 5′-TGATGGCACCAGTTGGTTGAC-3′ (21).

### B. Lower Primer

The reverse primer, called herein a lower primer is a 22-mer that hybridizes to the NA gene ending at position 805 and has a sequence of SEQ ID NO: 9 5′-GCATCAGGATAACAGGAGCAYTC-3′ In this case Y could be either C or T. Accordingly, the lower primer may comprise the sequences of SEQ ID NOS 51 or 52.

C. Amplicon Made by this Primer Set

The amplicon made by this primer set has the sequence of SEQ ID NO: 7 TGATGGCACCAGTTGGTTGACAATTGGAATTTCTGGCCCAGACAATGGGGCT GTGGCTGTATTGAAATACAATGGCATAATAACAGACACTATCAAGAGTTGGA GGAATAACATACTGAGAACTCAAGAGTCTGAATGTGCATGTGTAAATGGCTC TTGCTTTACTGTAATGACTGACGGACCAAGTAATGGTCAGGCATCACATAAG ATCTTCAAAATGGAAAAAGGGAAAGTGGTTAAATCAGTCGAATTGGATGCTC CCAATTATCACTATGAGGARTGCTCCTGTTATCCTGATGC (300 bp).

### Primer Pair for NA Gene in H5N2

A specific primer pair was designed to detect NA gene, segment 6 of the H5N2 subtype of Influenza A virus by amplifying a 361 base pair portion of the NA gene from nucleotide 440 to nucleotide 774 of the NA gene segment. This Specific primer pair was designed to detect H5N2 subtype-NA gene as a confirmation of specificity.

### A. Upper Primer

The forward primer, also herein called an upper primer was a 22-mer that hybridises to the NA gene beginning at position 440 and has the sequence of SEQ ID NO: 14 5′-AATGAGTTGGGTGTTCCGTTTC-3′.

### B. Lower Primer

The reverse primer, called herein a lower primer was a 27-mer that hybridizes to the NA gene ending at position 774 and has a sequence of SEQ ID NO: 15 5′-AACAGGAACATTCCTCTATATGCTGAG-3′.

C. Amplicon Made by this Primer Set

The amplicon made by this primer set has the sequence of SEQ ID NO: 13 AATGAGTTGGGTGTTCCGTTTCACTTGGGAACCAAACAGTGTGCATAGCAT GGTCCAGTTCAAGTTGCCATGACGGGAAAGCATGGTTGCACGTCTGTGTTAC TGGGGATGATAGAAATGCGACTGCTAGTTTCATTTATGATGGGATGCTCGTT GACAGTATAGGTTCATGGTCTCAAAATATCCTCAGAACTCAGGAGCCAGAGT GCGTTTGCATCAATGGGACTTGTACAGTAGTAATGACTGATGGAAGCGCATC AGGGAAAGCCGACACTAGAATATTATTCATTGAAGAGGGGAAAGTTGTTCAC ATTAGCCCATTGTCGGGAAGTGCTCAGCATATAGAGGAATGTTCCTGTT (361 bp).

### Example 4

### Detection Kits

In order to carry out the present the invention, Kit 1 was used for detection by suspension array technology and Kit 2 was used for PCR.

### Kit 1 for Use in Suspension Array Technology

### Detection of Generic Influenza A Virus

For general detecting and determination of the presence of Influenza A virus in a sample, a kit comprising microbeads immobilized with at least one nucleic acid sequence of SEQ ID N021 and/or 22 (MP-168 Upper and Lower probes common to all Influenza subtypes) was provided.

For sub-typing of the H5N1 subtype, the kit may further comprise at least one sequence selected from the group of H5N1 specific sequences. This group may consist of SEQ ID NOS:8 and 9 (probes/primers for the NA gene in H5N1 subtypes), SEQ ID NOS:16 and 17 (probes/primers for the HA region in H5N1 subtype) and SEQ ID NO: 18 and 19 (probe/primers for the HA-195 region in H5N1 subtypes) and SEQ ID NO:20 (probe for amplicon amplified by SEQ ID NOS:18 and 19). Accordingly, the kit may comprise at least one oligonucleotide comprising the sequences of SEQ ID NOS:30-60.

The kit may optionally comprise a suitable dye or label for quantitation by the Luminex system and information pertaining to use of the kit.

Kit 2 for Use with PCR Technology
Detection of Generic Influenza A virus with H5N1 Confirmation
Institute of Molecular and Cell Biology (IMCB), Flu A detection kit-2

### RT-PCR Diagnostic Kit

### Store at −20° C. in

100 Reactions/Kit, Lot 1, serial #001

### Non-Frost-Free Freezer

### Product Description

This Reverse Transcriptase-Polymerase Chain Reaction (RT-PCR) kit is made to detect the presence of generic influenza A virus with and H5N1 confirmation. The first strand cDNA reaction was carried out using the following reagents at the indicated concentrations.

This kit used three sets of primers: 1) HA primer pair specific to H5N1, 2) M primer pair for all Influenza A and 3) NA primer pair specific for H5N1. Amplified products were detected by ethidium bromide staining of amplified products after agarose gel electrophoresis.

The entire procedure was performed in one step.

Qiagen OneStep RT-PCR Kit [Cat. no. 210210] was used.

### Components

This kit comprises the following 2 tubes:

| | | Storage |
|---|---|---|
| | | Conditions |
| Tube | | (Short, |
| No. | Component | Long) |
| Tube 1 | Primer Mix comprising all | −20° C. |
| | three sets of primers. * | |
| Tube 2 | cDNA Positive Control | 4°^{.}, −20° C. |
| | (10-50 copies ea/μl) | |

RNase inhibitor included. Primers comprise nucleic acids of SEQ ID NOS: 2, 3, 8, 9, 11 and 12.

### Protocol for One Step RT-PCR

### 1. Sample Preparation

In a RNase-Free Eppendorf tube (0.5 ml or 0.2 ml size), the following reagents were added per test/per reaction:

| Tube No. | Description | 50 μl/Rxn | 20 μl/Rxn |
|---|---|---|---|
| Tube 1 | Primer Mix | 2.0 μl | 0.8 μl |
| | (3 primer sets) | | |
| From | Qiagen 5x buffer | 10.0 μl | 4.0 μl |
| Qiagen | Qiagen dNTP Mix | 2.0 μl | 0.8 μl |
| Kit | Qiagen Enzyme Mix | 2.0 μl | 0.8 μl |
| | RNase-free Water | 29.0 μl | 11.6 μl |
| Tube 2 or | RNA Sample | 5.0 μl | 2.0 μl |
| test sample | | | |
| | Total Volume | 50.0 μl | 20.0 μl |

To validate the results, a positive and negative control were included. The positive control being in the form of a cDNA comprising SEQ ID NOS 1, 7 and 10 and the negative control being any RNA isolated from a non-influenza type virus.

### 2. Thermal Cycling Protocol—A

Thermal cycling conditions for three-blocks type PCR cycler such as RoboCycler® by Stratagene:

| | Temp | | Num of | |
|---|---|---|---|---|
| Step | (° C.) | Duration | Cycle(s) | Step |
| 1 | 60 | 30 mins | 1 | Reverse |
| | | | | transcription |
| 2 | 95 | 15 mins | 1 | Initial |
| | | | | denaturation |
| 3 | 95 | 45 secs | 42 | Denaturation |
| | 59 | 76 secs | | Annealing |
| | 72 | 45 secs | | Extention |
| 4 | 72 | 3 mins | 1 | Final Extention |

### 3. Thermal Cycling Protocol—B

Thermal cycling conditions for one-block type PCR cycler such as Px2 Thermal Cycler by Thermo Electron.

| | Temp | | No. of | |
|---|---|---|---|---|
| Step | (° C.) | Duration | Cycle(s) | Step |
| 1 | 60 | 30 mins | 1 | Reverse |
| | | | | transcription |
| 2 | 95 | 15 mins | 1 | Initial |
| | | | | denaturation |
| 3 | 95 | 15 secs | 42 | Denaturation |
| | 59 | 25 secs | | Annealing |
| | 72 | 24 secs | | Extention |
| 4 | 72 | 3 mins | 1 | Final Extention |

### 4. Termination of PCR Reaction

(This step is optional).
(1) Add 30 ul of Chloroform/Tube. Vortex mix for 5 secs
(2) Centrifuge for 2 mins. (Top=Aqueous phase, Bottom=Organic phase)

### 5. Electrophoresis

The above product was resolved by DNA gel electrophoresis.

(1) DNA electrophoresis with 3% agarose.gel
(2) Use voltage, for example, at 100V for 30 mins.

### 6. Validating Results

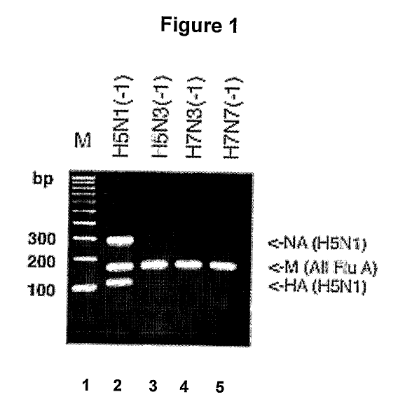

The expected product size was 114 bp for HA, 168 bp for the Matrix protein and 300 bp for NA are shown in FIG. 1.

Kit 3 for Use with PCR Technology

### Detection of Influenza a H5N1 Virus

Institute of Molecular and Cell Biology (IMCB), Flu A detection kit-3

### RT-PCR Diagnostic Kit

### Store at −20° C. in

100 Reactions/Kit, Lot 1, serial # 001

### Non-Frost-Free Freezer

### Product Description

This Reverse Transcriptase-Polymerase Chain Reaction (RT-PCR) kit is made to detect the presence of influenza A subtype H5N1 viral RNA. The first strand cDNA reaction was carried out using the following reagents at the indicated concentrations.

This kit uses two sets of primers, 1) HA primer pair specific to H5, and 2) NA primer pair specific for N1. Amplified products were detected by ethidium bromide staining of amplified products after agarose gel electrophoresis.

The entire procedure was performed in one step.

Qiagen OneStep RT-PCR Kit [Cat. no. 210210] was provided.

### Components

This kit consists of following 2 tubes:

| | | Storage |
|---|---|---|
| Tube | | Conditions |
| No. | Component | (Short, Long) |
| Tube 1 | Primer Mix containing | −20° C. |
| | all two sets of primers.* | |
| Tube 2 | cDNA Positive Control | 4°^{.}, −20° C. |
| | (50 copies ea./μl) | |

*RNase Inhibitor included. Primers Include SEQ ID NOS: 4, 5, 8 and 9. Protocol, One Step RT-PCR

### 7. Sample Preparation

In a RNase-Free Eppendorf tube (0.5 ml or 0.2 ml size), add the following reagents per test/per reaction:

| Tube No. | Description | 50 μl/Rxn | 20 μl/Rxn |
|---|---|---|---|
| Tube 1 | Primer Mix | 2.0 μl | 0.8 μl |
| | (2 primer sets) | | |
| From | Qiagen 5x buffer | 10.0 μl | 4.0 μl |
| Qiagen | Qiagen dNTP Mix | 2.0 μl | 0.8 μl |
| Kit | Qiagen Enzyme Mix | 2.0 μl | 0.8 μl |
| | RNase-free Water | 29.0 μl | 11.6 μl |
| Tube 2 or | RNA Sample | 5.0 μl | 2.0 μl |
| test sample | | | |
| | Total Volume | 50.0 μl | 20.0 μl |

*Be very careful of contamination.

To validate the results, a positive and negative control were included. The positive control being in the form of a cDNA comprising SEQ ID NOS 1, 7 and 10 and the negative control being any RNA isolated from a non-influenza type virus.

### Thermal Cycling Protocol-A

Thermal cycling conditions for three-blocks type PCR cycler such as RoboCycler® by Stratagene:

| | Temp | | Num of | |
|---|---|---|---|---|
| Step | (° C.) | Duration | Cycle(s) | Step |
| 1 | 60 | 30 mins | 1 | Reverse |
| | | | | transcription |
| 2 | 95 | 15 mins | 1 | Initial |
| | | | | denaturation |
| 3 | 95 | 45 secs | 42 | Denaturation |
| | 59 | 76 secs | | Annealing |
| | 72 | 45 secs | | Extention |
| 4 | 72 | 3 mins | 1 | Final Extention |

### Thermal Cycling Protocol-B

Thermal cycling conditions for one-block type PCR cycler such as Px2 Thermal Cycler by Thermo Electron.

| | Temp | | Num of | |
|---|---|---|---|---|
| Step | (° C.) | Duration | Cycle(s) | Step |
| 1 | 60 | 30 mins | 1 | Reverse |
| | | | | transcription |
| 2 | 95 | 15 mins | 1 | Initial |
| | | | | denaturation |
| 3 | 95 | 15 secs | 42 | Denaturation |
| | 59 | 25 secs | | Annealing |
| | 72 | 24 secs | | Extention |
| 4 | 72 | 3 mins | 1 | Final Extention |

### Termination of PCR Reaction

(This step is optional).
(1) Add 30 μl of Chloroform/Tube. Vortex mix for 5 secs
(2) Centrifuge for 2 mins. (Top=Aqueous phase, Bottom=Organic phase)

### Electrophoresis

Resolve the above product by DNA gel electrophoresis.

(1) DNA electrophoresis with 3% agarose.gel
(2) Use voltage, for example, at 100V for 30 mins.

### Validating Results

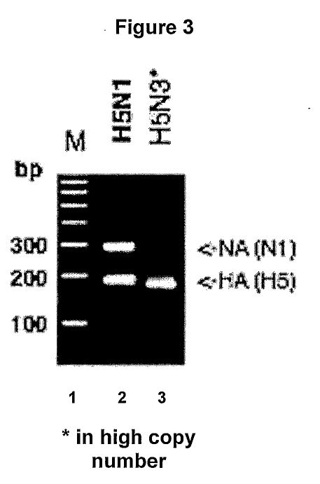

The expected product size of HA band was 195 bp for H5N1 and approximately 183 bp for H5N3. Thus the two subtypes may be differentiated and the expected product size of NA band is 300 bp for H5N1 as shown in FIG. 3.

A kit according to this example was typically prepared to contain 50 or 100 reactions. The kit was stored at −20° C. in a non-frost-free freezer. FIG. 1 shows results that are obtained using Kit 2 and FIG. 3 shows results that are obtained using Kit 3.

The Reverse Transcriptase-Polymerase Chain Reaction (RT-PCR) kits described herein may be used for detecting the presence of H5N1 subtype of influenza A (avian flu) RNA in samples extracted from specimens with an appropriate RNA extraction method of choice. The kits are optimized to detect a few molecules of the viral RNA in 5 μl of test sample and the entire procedure is performed in one step.

### Electrophoresis

The products of the PCR reactions were resolved by DNA gel electrophoresis by using 5 μl of the product reaction mixture product per lane. 3% agarose gel provided good resolution; gels were run at 100 V for 30 min.

### Example 5

### Specificity of RT-PCR Using the Primers

To verify that the primer sets designed in Kit 2 and Kit 3 as described in Example 4 may be used to detect H5N1 subtype of influenza A virus specifically. The amplification of selected viruses was tested by RT-PCR using the primer sets of SEQ ID NO: 2, 3, 8, 9, and 11 and 12 for Kit 2 and primer sets SEQ ID NOS: 4, 5, 8 and 9 for Kit 3. The following influenza A virus subtypes were tested at the indicated titer to check the specificity of the primer sets:

### H5N1 (−1) A/chicken/Vietnam/8/2004 (HPAI),

H5N3 (−1) A tern/Australia/75/(LPAI),

### H7N3 (−1) A/chicken/queensland/1994 (HPAI) and

### H7N7 (−1) A/duck/Victoria/1976 (LPAI).

The results in FIGS. 1 and 3 show the primer pair specificity to the H5N1 subtype of influenza A. Thus, it is demonstrated that the primer pairs are highly specific to detection of the H5N1 subtype of influenza A.

### Example 6

### Analysis of Human Patient Samples

Clinical samples were obtained from a number of human patients and were analyzed by the assay method of the present invention, using primer sets of Kit 2 or Kit 3.

### PCR Diagnostic Kit (RT-PCR)

A kit of this example is typically prepared to contain 50 or 100 reactions. This Real-Time Reverse Transcriptase-Polymerase Chain Reaction kit is optimized to detect the presence of the H5N1 subtype of influenza A RNA in a biological sample. This kit is optimized for use with the Applied Biosystems Real-Time PCR, ABI Prism 7500, but is not limited to this system. The kit may also be used with other suitable detection platforms as described elsewhere herein.

The kit is sufficiently sensitive to detect a few molecules of RNA in each RT-PCR reaction.

### Components

This kit of this Example consists of the following 4 tubes:

Tube 1: Reaction Mix (e.g. ABI cat. No. 4309169)
Tube 2: Enzyme Mix (e.g. ABI cat. No. 4309169)
Tube 3: Probe Mix (3 μM upper primer (SEQ ID NOS: 2, 4, 8, 11, and or 14), 3 μM lower primer (SEQ ID NOS: 3, 5, 9, 12 and or 15) and 2 μM probe (SEQ ID NO: 1) in 20 mM Tris, 1 mM EDTA pH 8.2)
Tube 4: Positive Control (RNA transcripts of the gene targeted by the primers)

### Protocol

### 1. RT-PCR

The following reaction mix is prepared in a 96-well optical plate:

| Tube No. | Description | Vol/Rxn |
|---|---|---|
| Tube 1 | Reaction Mix | 25.0 μl |
| Tube 2 | Enzyme Mix | 1.25 μl |
| Tube 3 | Probe Mix | 5.0 μl |
| Tube 4 | Distilled Water | 13.75 μl |
| — | RNA Sample | 5.0 μl |
| | Total Volume | 50.0 μl |

Caution should be taken to avoid contamination.

### 2. Thermal Cycling Conditions

| Step | Temp (° C.) | Duration | No. of Cycle(s) |
|---|---|---|---|
| 1 | 48 | 30 mins | 1 |
| 2 | 95 | 10 mins | 1 |
| 3 | 95 | 15 secs | 50 |
| 4 | 60 | 60 secs | 1 |

The primer set and probe are tested for their ability to detect the H5N1 subtype of influenza A using the Stratagene real-time PCR system Mx3000P. The system is used according to the manufacturer's instructions on samples from infected patients. The samples are diluted several fold to a total of viral copy number per 5 μl ranging between 7.5 to 6.

The results demonstrate that the primer set and probe provide a sensitive and specific assay for the H5N1 subtype of influenza A that is useful in a clinical setting.

### Results for Example 6

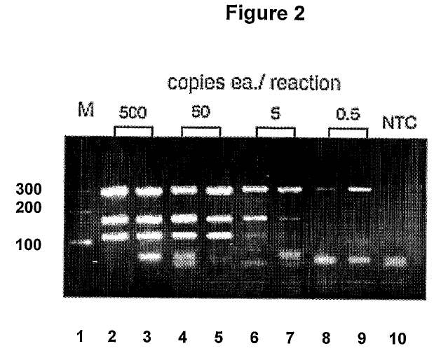

FIG. 2 demonstrates the sensitivity of the primers at differing concentrations achieved using the primers for the detection of Influenza A subtype H5N1 where the virus copy number per sample loaded varies from 500 copies per reaction to 0.5 copies per reaction. Lane 1 is a marker, lanes 2 & 3 contain 500 copies of the virus per 5 μl, lanes 4 & 5 contain 50 copies of the virus per 5 μl, lanes 6 & 7 contain 5 copies of the virus per 5 μl, lanes 8 & 9 contain 0.5 copies of the virus per 5 μl, lane 10 contains a negative control of an unrelated virus. Positive DNA controls were prepared from individually cloned DNA derived from corresponding amplicons.

Although the present invention has been described in detail with reference to examples above, it is understood, that various modifications may be made without departing from the spirit of the invention. All cited patents, patent applications and publications referred to in this application is herein incorporated by reference in their entirety.

1-72. (canceled)

73. An isolated oligonucleotide comprising at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 22, SEQ ID NO: 25, SEQ ID NO: 26, SEQ ID NO: 28, SEQ ID NO: 29, SEQ ID NO:30, and SEQ ID NOs: 38-44.

74. An isolated oligonucleotide consisting of at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28 and SEQ ID NO: 29.

75. The isolated oligonucleotide according to claim 73, wherein the oligonucleotide comprising SEQ ID NO:22 is an oligonucleotide comprising at least one nucleotide sequence selected from the group consisting of SEQ ID NO:32 and SEQ ID NO:33; the oligonucleotide comprising SEQ ID NO:25 is an oligonucleotide comprising at least one nucleotide sequence selected from the group consisting of SEQ ID NO:16 and SEQ ID NO:34; the oligonucleotide comprising SEQ ID NO:26 is an oligonucleotide comprising at least one nucleotide sequence selected from the group consisting of SEQ ID NO:17, SEQ ID NO:35 and SEQ ID NO:36; the oligonucleotide comprising SEQ ID NO:28 is an oligonucleotide comprising at least one nucleotide sequence selected from the group consisting of SEQ ID NO:19 and SEQ ID NOs:45 to 48; and/or the oligonucleotide comprising SEQ ID NO:29 is an oligonucleotide comprising at least one nucleotide sequence selected from the group consisting of SEQ ID NO:20 and SEQ ID NOs:49 to 50.

76. The isolated oligonucleotide according to claim 74, wherein the oligonucleotide consisting of SEQ ID NO:23 is an oligonucleotide comprising at least one nucleotide sequence selected from the group consisting of SEQ ID NO:21, SEQ ID NO:30 and SEQ ID NO:31; the oligonucleotide consisting of SEQ ID NO:24 is an oligonucleotide comprising at least one nucleotide sequence selected from the group consisting of SEQ ID NO:22, SEQ ID NO:32 and SEQ ID NO:33; the oligonucleotide consisting of SEQ ID NO:25 is an oligonucleotide comprising at least one nucleotide sequence selected from the group consisting of SEQ ID NO:16 and SEQ ID NO:34; the oligonucleotide consisting of SEQ ID NO:26 is an oligonucleotide comprising at least one nucleotide sequence selected from the group consisting of SEQ ID NO:17, SEQ ID NO:35 and SEQ ID NO:36; the oligonucleotide consisting of SEQ ID NO:27 is an oligonucleotide comprising at least one nucleotide sequence selected from the group consisting of SEQ ID NO:18 and SEQ ID NOs:37 to 44; the oligonucleotide consisting of SEQ ID NO:28 is an oligonucleotide comprising at least one nucleotide sequence selected from the group consisting of SEQ ID NO:19 and SEQ ID NOs:45 to 48; and/or the oligonucleotide consisting of SEQ ID NO:29 is an oligonucleotide comprising at least one nucleotide sequence selected from the group consisting of SEQ ID NO:20 and SEQ ID NOs:49 to 50.

77. A method of determining the presence of an H5N1 subtype of Influenza A virus in a biological sample, the method comprising the steps of:
(a) providing a biological sample;
(b) contacting at least one oligonucleotide with at least one nucleic acid in the biological sample, or contacting the oligonucleotide with at least one nucleic acid extracted, purified and/or amplified from the biological sample, wherein the oligonucleotide comprises at least one nucleotide sequence selected from the group consisting of:
SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 28, SEQ ID NO: 29, and SEQ ID NOs: 38-44;
and
(c) detecting any binding resulting from the contacting in step (b), whereby the detection of the binding indicates the presence of an H5N1 subtype of an Influenza A virus.

78. A method of determining the presence of an H5N1 subtype of Influenza A virus in a biological sample, the method comprising the steps of:
(a) providing a biological sample;
(b) contacting at least one oligonucleotide with at least one nucleic acid in the biological sample, or contacting the oligonucleotide with at least one nucleic acid extracted, purified and/or amplified from the biological sample, wherein the oligonucleotide consists of at least one nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29;
and
(c) detecting any binding resulting from the contacting in step (b), whereby the detection of the binding indicates the presence of an H5N1 subtype of an Influenza A virus.

79. The method according to claim 77, wherein the oligonucleotide comprising SEQ ID NO:23 is an oligonucleotide comprising at least one nucleotide sequence selected from the group consisting of SEQ ID NO:21, SEQ ID NO:30 and SEQ ID NO:31; the oligonucleotide comprising SEQ ID NO:24 is an oligonucleotide comprising at least one nucleotide sequence selected from the group consisting of SEQ ID NO:22, SEQ ID NO:32 and SEQ ID NO:33; the oligonucleotide comprising SEQ ID NO:25 is an oligonucleotide comprising at least one nucleotide sequence selected from the group consisting of SEQ ID NO:16 and SEQ ID NO:34; the oligonucleotide comprising SEQ ID NO:26 is an oligonucleotide comprising at least one nucleotide sequence selected from the group consisting of SEQ ID NO:17, SEQ ID NO:35 and SEQ ID NO:36; the oligonucleotide comprising SEQ ID NO:28 is an oligonucleotide comprising at least one nucleotide sequence selected from the group consisting of SEQ ID NO:19 and SEQ ID NOs:45 to 48; and/or the oligonucleotide consisting of SEQ ID NO:29 is an oligonucleotide comprising at least one nucleotide sequence selected from the group consisting of SEQ ID NO:20 and SEQ ID NOs:49 to 50.

80. The method according to claim 78, wherein the oligonucleotide consisting of SEQ ID NO:23 is an oligonucleotide comprising at least one nucleotide sequence selected from the group consisting of SEQ ID NO:21, SEQ ID NO:30 and SEQ ID NO:31; the oligonucleotide consisting of SEQ ID NO:24 is an oligonucleotide comprising at least one nucleotide sequence selected from the group consisting of SEQ ID NO:22, SEQ ID NO:32 and SEQ ID NO:33; the oligonucleotide consisting of SEQ ID NO:25 is an oligonucleotide comprising at least one nucleotide sequence selected from the group consisting of SEQ ID NO:16 and SEQ ID NO:34; the oligonucleotide consisting of SEQ ID NO:26 is an oligonucleotide comprising at least one nucleotide sequence selected from the group consisting of SEQ ID NO:17, SEQ ID NO:35 and SEQ ID NO:36; the oligonucleotide consisting of SEQ ID NO:27 is an oligonucleotide comprising at least one nucleotide sequence selected from the group consisting of SEQ ID NO:18 and SEQ ID NOs:37 to 44; the oligonucleotide consisting of SEQ ID NO:28 is an oligonucleotide comprising at least one nucleotide sequence selected from the group consisting of SEQ ID NO:19 and SEQ ID NOs:45 to 48; and/or the oligonucleotide consisting of SEQ ID NO:29 is an oligonucleotide comprising at least one nucleotide sequence selected from the group consisting of SEQ ID NO:20 and SEQ ID NOs:49 to 50.

81. The method according to claim 77, wherein the oligonucleotides are probes and the method comprises:
(i) providing a biological sample;
(ii) labeling the at least one nucleic acid in the biological sample or extracted, purified or amplified from the sample with at least one reporter label;
(iii) immobilizing at least one probe to at least one microbead comprising at least one fluorescent dye;
(iv) contacting the at least one probe with the at least one nucleic acid to allow binding of the probe(s) and nucleic acid(s);
(v) identifying microbeads based on the fluorescent intensity of the at least one fluorescent dye with a first laser light and detecting binding of nucleic acid(s) to probe(s) immobilized on identified microbead(s) with a second laser light based on the reporter label(s);
whereby the detection of binding of the nucleic acid(s) to probe(s) indicates the presence of the H5N1 subtype of Influenza A virus.

82. The method according to claim 81, wherein the labeling of the at least one nucleic acid in step (ii) is done after the contacting in step (iv).

83. The method according to claim 81, wherein the step (c) of detecting is carried out by using Suspension Array Technology.

84. The method according to claim 77, wherein the contacting in step (b) comprises contacting at least two oligonucleotides forming a primer pair to the nucleic acid and the step (c) of detecting is by a polymerase chain reaction.

85. The method according to claim 84, wherein the primer pair binds to the nucleic acid(s) and amplify at least one amplicon comprising the sequence of SEQ ID NO:6, the primer pair comprising at least one forward primer comprising the nucleotide sequence SEQ ID NO:25 and at least one reverse primer comprising the nucleotide sequence SEQ ID NO:26.

86. The method according to claim 85, wherein the forward primer comprises the nucleotide sequence of SEQ ID NO:34 and/or the reverse primer comprises at least one nucleotide sequence selected from the group consisting of SEQ ID NO:35 and SEQ ID NO:36.

87. The method according to claim 84, wherein the primer pair binds to the nucleic acid(s) and amplify at least one amplicon comprising the sequence of SEQ ID NO:7, the primer pair comprising at least one forward primer comprising the nucleotide sequence SEQ ID NO:18 and at least one reverse primer comprising the nucleotide sequence SEQ ID NO:19.

88. The method according to claim 87, wherein the forward primer comprises at least one nucleotide sequence selected from the group consisting of SEQ ID NOs:37-44 and/or the reverse primer comprises at least one nucleotide sequence selected from the group consisting of SEQ ID NOs:45-48.

89. The method according to claim 87 wherein a probe comprising a nucleotide sequence selected from the group consisting of SEQ ID NOs:49 and 50 is capable of binding to the amplicon.

90. The method according to claim 78, wherein the oligonucleotides are probes and the method comprises:
(i) providing a biological sample;
(ii) labeling the at least one nucleic acid in the biological sample or extracted, purified or amplified from the sample with at least one reporter label;
(iii) immobilizing at least one probe to at least one microbead comprising at least one fluorescent dye;
(iv) contacting the at least one probe with the at least one nucleic acid to allow binding of the probe(s) and nucleic acid(s);
(v) identifying microbeads based on the fluorescent intensity of the at least one fluorescent dye with a first laser light and detecting binding of nucleic acid(s) to probe(s) immobilized on identified microbead(s) with a second laser light based on the reporter label(s);
whereby the detection of binding of the nucleic acid(s) to probe(s) indicates the presence of the H5N1 subtype of Influenza A virus.

91. The method according to claim 90, wherein the labeling of the at least one nucleic acid in step (ii) is done after the contacting in step (iv).

92. The method according to claim 90, wherein the step (c) of detecting is carried out by using Suspension Array Technology.

93. The method according to claim 78, wherein the contacting in step (b) comprises contacting at least two oligonucleotides forming a primer pair to the nucleic acid and the step (c) of detecting is by a polymerase chain reaction.

94. The method according to claim 93, wherein the primer pair binds to the nucleic acid(s) and amplify at least one amplicon comprising the sequence of SEQ ID NO:6, the primer pair comprising at least one forward primer comprising the nucleotide sequence SEQ ID NO:25 and at least one reverse primer comprising the nucleotide sequence SEQ ID NO:26.

95. The method according to claim 94, wherein the forward primer comprises the nucleotide sequence of SEQ ID NO:34 and/or the reverse primer comprises at least one nucleotide sequence selected from the group consisting of SEQ ID NO:35 and SEQ ID NO:36.

96. The method according to claim 93, wherein the primer pair binds to the nucleic acid(s) and amplify at least one amplicon comprising the sequence of SEQ ID NO:7, the primer pair comprising at least one forward primer comprising the nucleotide sequence SEQ ID NO:18 and at least one reverse primer comprising the nucleotide sequence SEQ ID NO:19.

97. The method according to claim 96, wherein the forward primer comprises at least one nucleotide sequence selected from the group consisting of SEQ ID NOs:37-44 and/or the reverse primer comprises at least one nucleotide sequence selected from the group consisting of SEQ ID NOs:45-48.

98. The method according to claim 96 wherein a probe comprising a nucleotide sequence selected from the group consisting of SEQ ID NOs:49 and 50 is capable of binding to the amplicon.

99. A kit for the detection of Influenza A virus, the kit comprising at least one oligonucleotide comprising a nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO:22, SEQ ID NO: 25, SEQ ID NO: 26, SEQ ID NO: 28, SEQ ID NO: 29, SEQ ID NO: 30, and SEQ ID NOs: 38-44.

100. A kit for the detection of Influenza A virus, the kit comprising at least one oligonucleotide consisting of a nucleotide sequence selected from the group consisting of: SEQ ID NO:4, SEQ ID NO:5, SEQ ID NO:6, SEQ ID NO:7, SEQ ID NO:8, SEQ ID NO:9, SEQ ID NO: 23, SEQ ID NO: 24, SEQ ID NO: 25, SEQ ID NO: 26, SEQ ID NO: 27, SEQ ID NO: 28, SEQ ID NO: 29.

101. The kit according to claim 99, wherein the oligonucleotide comprising SEQ ID NO:22 is an oligonucleotide comprising at least one nucleotide sequence selected from the group consisting of SEQ ID NO:32 and SEQ ID NO:33; SEQ ID NO:25 is an oligonucleotide comprising at least one nucleotide sequence selected from the group consisting of SEQ ID NO:16 and SEQ ID NO:34; the oligonucleotide comprising SEQ ID NO:26 is an oligonucleotide comprising at least one nucleotide sequence selected from the group consisting of SEQ ID NO:17, SEQ ID NO:35 and SEQ ID NO:36; the oligonucleotide comprising SEQ ID NO:28 is an oligonucleotide comprising at least one nucleotide sequence selected from the group consisting of SEQ ID NO:19 and SEQ ID NOs:45 to 48; and/or the oligonucleotide comprising SEQ ID NO:29 is an oligonucleotide comprising at least one nucleotide sequence selected from the group consisting of SEQ ID NO:20 and SEQ ID NOs:49 to 50.

102. The kit according to claim 100, wherein the oligonucleotide consisting of SEQ ID NO:23 is an oligonucleotide comprising at least one nucleotide sequence selected from the group consisting of SEQ ID NO:21, SEQ ID NO:30 and SEQ ID NO:31; the oligonucleotide consisting of SEQ ID NO:24 is an oligonucleotide comprising at least one nucleotide sequence selected from the group consisting of SEQ ID NO:22, SEQ ID NO:32 and SEQ ID NO:33; the oligonucleotide consisting of SEQ ID NO:25 is an oligonucleotide comprising at least one nucleotide sequence selected from the group consisting of SEQ ID NO:16 and SEQ ID NO:34; the oligonucleotide consisting of SEQ ID NO:26 is an oligonucleotide comprising at least one nucleotide sequence selected from the group consisting of SEQ ID NO:17, SEQ ID NO:35 and SEQ ID NO:36; the oligonucleotide consisting of SEQ ID NO:27 is an oligonucleotide comprising at least one nucleotide sequence selected from the group consisting of SEQ ID NO:18 and SEQ ID NOs:37 to 44; the oligonucleotide consisting of SEQ ID NO:28 is an oligonucleotide comprising at least one nucleotide sequence selected from the group consisting of SEQ ID NO:19 and SEQ ID NOs:45 to 48; and/or the oligonucleotide consisting of SEQ ID NO:29 is an oligonucleotide comprising at least one nucleotide sequence selected from the group consisting of SEQ ID NO:20 and SEQ ID NOs: 49 to 50.


SEQUENCE LISTING (60 sequences)

SEQ ID NO: 1 - DNA, 114 bp, Influenza A virus
  misc_feature: HA segment 4
caaacagattagtccttgcgactgggctcagaaatagccctcaaagagagagaagaagaaaaaagagaggactatttggagctatagcaggttttatagagggaggatggcarg

SEQ ID NO: 2 - DNA, 24 bp, Artificial Sequence
  Synthesized primer
caaacagattagtccttgcgactg

SEQ ID NO: 3 - DNA, 22 bp, Artificial Sequence
  Synthesized primer
cytgccatcctccctctataaa

SEQ ID NO: 4 - DNA, 21 bp, Artificial Sequence
  Synthesized primer
gccattccacaayatacaccc

SEQ ID NO: 5 - DNA, 24 bp, Artificial Sequence
  Synthesized primer
tacccataccaaccatctaccatt

SEQ ID NO: 6 - DNA, 114 bp, Influenza A virus; identical to SEQ ID NO: 1
  misc_feature: r is a or g

SEQ ID NO: 7 - DNA, 300 bp, Influenza A virus
  misc_feature: r is a or g
tgatggcaccagttggttgacaattggaatttctggcccagacaatggggctgtggctgtattgaaatacaatggcataataacagacactatcaagagttggaggaataacatactgagaactcaagagtctgaatgtgcatgtgtaaatggctcttgctttactgtaatgactgacggaccaagtaatggtcaggcatcacataagatcttcaaaatggaaaaagggaaagtggttaaatcagtcgaattggatgctcccaattatcactatgaggartgctcctgttatcctgatgc

SEQ ID NO: 8 - DNA, 21 bp, Artificial Sequence
  Synthesized primer
tgatggcaccagttggttgac

SEQ ID NO: 9 - DNA, 23 bp, Artificial Sequence
  Synthesized primer
gcatcaggataacaggagcaytc

SEQ ID NO: 10 - DNA, 168 bp, Influenza A virus
gagtcttctaaccgaggtcgaaacgtacgttctctctatcatcccgtcaggccccctcaaagccgagatcgcgcagaaacttgaagatgtctttgcaggaaagaacaccgatctcgaggctctcatggagtggctaaagacaagaccaatcctgtcacctctgactaa

SEQ ID NO: 11 - DNA, 24 bp, Artificial Sequence
  Synthesized primer
gagtcttctaaccgaggtcgaaac

SEQ ID NO: 12 - DNA, 25 bp, Artificial Sequence
  Synthesized primer
ttagtcagaggtgacaggattggtc

SEQ ID NO: 13 - DNA, 361 bp, Influenza A virus
aatgagttgggtgttccgtttcacttgggaaccaaacaagtgtgcatagcatggtccagttcaagttgccatgacgggaaagcatggttgcacgtctgtgttactggggatgatagaaatgcgactgctagtttcatttatgatgggatgctcgttgacagtataggttcatggtctcaaaatatcctcagaactcaggagccagagtgcgtttgcatcaatgggacttgtacagtagtaatgactgatggaagcgcatcagggaaagccgacactagaatattattcattgaagaggggaaagttgttcacattagcccattgtcgggaagtgctcagcatatagaggaatgttcctgtt

SEQ ID NO: 14 - DNA, 22 bp, Artificial Sequence
  Synthesized primer
aatgagttgggtgttccgtttc

SEQ ID NO: 15 - DNA, 27 bp, Artificial Sequence
  Synthesized primer
aacaggaacattcctctatatgctgag

SEQ ID NO: 16 - DNA, 24 bp, Artificial Sequence
  HA-114 Upper Probe/Primer
caaacarattartycttgcdacwg

SEQ ID NO: 17 - DNA, 22 bp, Artificial Sequence
  HA-114 Lower Probe/Primer
cctgccatcctccytctataaa

SEQ ID NO: 18 - DNA, 21 bp, Artificial Sequence
  HA-195 Upper Probe/Primer
gccattccacaayatmcaycc

SEQ ID NO: 19 - DNA, 24 bp, Artificial Sequence
  HA-195 Lowr Probe/Primer
tayccataccaaccatctaycatt

SEQ ID NO: 20 - DNA, 22 bp, Artificial Sequence
  HA-195 Probe
grcattccccgatggtgagagg

SEQ ID NO: 21 - DNA, 24 bp, Artificial Sequence
  MP-168 Upper Probe/Primer
gagycttctaaccgaggtcgaaac

SEQ ID NO: 22 - DNA, 25 bp, Artificial Sequence
  MP-168 Lower Probe/Primer
ttagtcagaggtgacagrattggtc

SEQ ID NO: 23 - DNA, 24 bp, Artificial Sequence
  MP-168 Upper Variable
gagncttctaaccgaggtcgaaac

SEQ ID NO: 24 - DNA, 25 bp, Artificial Sequence
  MP-168 Lower Variable
ttagtcagaggtgacagnattggtc

SEQ ID NO: 25 - DNA, 24 bp, Artificial Sequence
  HA-114 Upper Variable
caaacanattantncttgcnacwg

SEQ ID NO: 26 - DNA, 22 bp, Artificial Sequence
  HA-114 Lower Variable
cctgccatcctccntctataaa

SEQ ID NO: 27 - DNA, 21 bp, Artificial Sequence
  HA-195 Upper Variable
gccattccacaanatncancc

SEQ ID NO: 28 - DNA, 24 bp, Artificial Sequence
  HA-195 Lower Variable
tanccataccaaccatctancatt

SEQ ID NO: 29 - DNA, 22 bp, Artificial Sequence
  HA-195 Probe Variable
gncattccccgatggtgagagg

SEQ ID NO: 30 - DNA, 24 bp, Artificial Sequence
  MP-165 Upper probe/primer var 1
gagccttctaaccgaggtcgaaac

SEQ ID NO: 31 - DNA, 24 bp, Artificial Sequence
  MP-165 Upper probe/primer var 2
gagtcttctaaccgaggtcgaaac

SEQ ID NO: 32 - DNA, 25 bp, Artificial Sequence
  MP-168 Lower probe/primer var 1
ttagtcagaggtgacagaattggtc

SEQ ID NO: 33 - DNA, 25 bp, Artificial Sequence
  MP-168 Lower probe/primer var 2
ttagtcagaggtgacaggattggtc

SEQ ID NO: 34 - DNA, 24 bp, Artificial Sequence
  HA-114 Upper probe/primer
caaacagattagtycttgcgactg

SEQ ID NO: 35 - DNA, 22 bp, Artificial Sequence
  MP-114 Lower probe/primer var 1
cctgccatcctccctctataaa

SEQ ID NO: 36 - DNA, 22 bp, Artificial Sequence
  HA-114 Lower probe/primer var 2
cctgccatcctccttctataaa

SEQ ID NO: 37 - DNA, 21 bp, Artificial Sequence
  HA-195 Upper probe/primer var 1
gccattccacaacatacaccc

SEQ ID NO: 38 - DNA, 21 bp, Artificial Sequence
  HA-195 Upper probe/primer var 2
gccattccacaatatacaccc

SEQ ID NO: 39 - DNA, 21 bp, Artificial Sequence
  HA-195 Upper probe/primer var 3
gccattccacaacatccaccc

SEQ ID NO: 40 - DNA, 21 bp, Artificial Sequence
  HA-195 Upper probe/primer var 4
gccattccacaatatccaccc

SEQ ID NO: 41 - DNA, 21 bp, Artificial Sequence
  HA-195 Upper probe/primer var 5
gccattccacaacatacatcc

SEQ ID NO: 42 - DNA, 21 bp, Artificial Sequence
  HA-195 Upper probe/primer var 6
gccattccacaatatacatcc

SEQ ID NO: 43 - DNA, 21 bp, Artificial Sequence
  HA-195 Upper probe/primer var 7
gccattccacaacatccatcc

SEQ ID NO: 44 - DNA, 21 bp, Artificial Sequence
  HA-195 Upper probe/primer var 8
gccattccacaatatccatcc

SEQ ID NO: 45 - DNA, 24 bp, Artificial Sequence
  HA-195 Lower probe/primer var 1
tacccataccaaccatctaccatt

SEQ ID NO: 46 - DNA, 24 bp, Artificial Sequence
  HA-195 Lower probe/primer var 2
tacccataccaaccatctatcatt

SEQ ID NO: 47 - DNA, 24 bp, Artificial Sequence
  HA-195 Lower probe/primer var 3
tatccataccaaccatctaccatt

SEQ ID NO: 48 - DNA, 24 bp, Artificial Sequence
  HA-195 Lower probe/primer var 4
tatccataccaaccatctatcatt

SEQ ID NO: 49 - DNA, 22 bp, Artificial Sequence
  HA-195 Probe var 1
gacattccccgatggtgagagg

SEQ ID NO: 50 - DNA, 22 bp, Artificial Sequence
  HA-195 Probe var 2
ggcattccccgatggtgagagg

SEQ ID NO: 51 - DNA, 23 bp, Artificial Sequence
  NA-300 Lower probe/primer var 1
gcatcaggataacaggagcactc

SEQ ID NO: 52 - DNA, 23 bp, Artificial Sequence
  NA-300 Lower probe/primer var 2
gcatcaggataacaggagcattc

SEQ ID NO: 53 - DNA, 21 bp, Artificial Sequence
  MP-168 probe
gctttgagggggcctganggn

SEQ ID NO: 54 - DNA, 21 bp, Artificial Sequence
  MP-168 probe var
gctttgagggggcctgayggr

SEQ ID NO: 55 - DNA, 195 bp, Influenza A subtype H5N1
  misc_feature: HA amplicon
gccattccacaacatacaccctctcaccatcggggaatgccccaaatatgtgaaatcaaacagattagtccttgcgactgggctcagaaatagccctcaaagagagagaagaagaaaaaagagaggactatttggagctatagcaggttttatagagggaggatggcagggaatggtagatggttggtatgggta

SEQ ID NO: 56 - DNA, 21 bp, Artificial Sequence
  MP-168 probe var 1
gctttgagggggcctgacgga

SEQ ID NO: 57 - DNA, 21 bp, Artificial Sequence
  MP-168 probe var 2
gctttgagggggcctgatgga

SEQ ID NO: 58 - DNA, 21 bp, Artificial Sequence
  MP-168 probe var 3
gctttgagggggcctgacggg

SEQ ID NO: 59 - DNA, 21 bp, Artificial Sequence
  MP-168 probe var 4
gctttgagggggcctgatggg

SEQ ID NO: 60 - DNA, 22 bp, Artificial Sequence
  NA-300 probe
acagccacagccccattgtctg